SEQUENCE LISTING (13 sequences)

SEQ ID NO: 1 - DNA, 102 bp, Human
tatgaattcatggcgcttttgttgaccacggtcattgctctcacttgccttggcggctttgcctccccaggccctgtgcctccctctacagccctcaggtac

SEQ ID NO: 2 - DNA, 101 bp, Human
gttgatgctccataccatgctgccattgcagagcggagccttctggttctgggtgatgttgaccagctcctcaatgaggtacctgagggctgtagagggag

SEQ ID NO: 3 - DNA, 100 bp, Human
ctctgggtcttctcgatggcactgcagcctgacacgttgatcagggattccagggctgcacagtacatgccagctgtcaggttgatgctccataccatgc

SEQ ID NO: 4 - DNA, 100 bp, Human
cctcgattttggtgtctcggacatgcaagctggaaaactgcccagctgagaccttgtgcgggcagaatccgctcagcatcctctgggtcttctcgatggc

SEQ ID NO: 5 - DNA, 96 bp, Human
tcggatcctcagttgaaccgtccctcgcgaaaaagtttctttaaatgtaagagcaggtcctttacaaactgggccacctcgattttggtgtctcgg

SEQ ID NO: 6 - DNA, 39 bp, Artificial Sequence
  IL13 312#3 mut5-3 primer
caacctgacagctggcatgtactgtgcagccctggaatc

SEQ ID NO: 7 - DNA, 39 bp, Artificial Sequence
  IL13 312#3 mut3-5 primer
gattccagggctgcacagtacatgccagctgtcaggttg

SEQ ID NO: 8 - DNA, 43 bp, Human
atctctagagccgccaccatgcttctcctggtgacaagccttc

SEQ ID NO: 9 - DNA, 34 bp, Human
gagggaggcacagggcctgggatcaggaggaatg

SEQ ID NO: 10 - DNA, 34 bp, Human
cattcctcctgatcccaggccctgtgcctccctc

SEQ ID NO: 11 - DNA, 35 bp, Human
gggaccatatttggactcgttgaaccgtccctcgc

SEQ ID NO: 12 - DNA, 35 bp, Human
gcgagggacggttcaacgagtccaaatatggtccc

SEQ ID NO: 13 - DNA, 26 bp, Human
atgcggccgctcagcgagggggcagg

(19) United States
(12) Patent Application Publication
(10) Pub. No.: US 2003/0171546 A1
(43) Pub. Date: Sep. 11, 2003

(54) Chimeric immunoreceptor useful in treating human cancers

(75) Inventor: Michael Jensen, Pasadena, CA (US)

Correspondence Address: ROTHWELL, FIGG, ERNST & MANBECK, P.C., 1425 K STREET, N.W., SUITE 800, WASHINGTON, DC, 20005, US

(73) Assignee: CITY OF HOPE, DUARTE, CA

(21) Appl. No.: 10/134,645

(22) Filed: Apr. 30, 2002

Related U.S. Application Data
(60) Provisional application No. 60/286,981, filed on Apr. 30, 2001.

(51) Int. Cl. C07K 14/705; C12P 21/02; C12N 5/06; C07K 14/52

(52) U.S. Cl. 530/350; 530/351; 435/69.1; 435/69.5; 435/320.1; 435/325

(57) ABSTRACT

The present invention relates to chimeric transmembrane immunoreceptors, named “zetakines,” comprised of an extracellular domain comprising a soluble receptor ligand linked to a support region capable of tethering the extracellular domain to a cell surface, a transmembrane region and an intracellular signalling domain. Zetakines, when expressed on the surface of T lymphocytes, direct T cell activity to those specific cells expressing a receptor for which the soluble receptor ligand is specific. Zetakine chimeric immunoreceptors represent a novel extension of antibody-based immunoreceptors for redirecting the antigen specificity of T cells, with application to treatment of a variety of cancers, particularly via the autocrin/paracrine cytokine systems utilized by human maligancy. In a preferred embodiment is a glioma-specific immunoreceptor comprising the extracellular targetting domain of the IL-13Rα2-specific IL-13 mutant IL-13(E13Y) linked to the Fc region of IgG, the transmembrane domain of human CD4, and the human CD3 zeta chain.

### TECHNICAL FIELD

This invention relates to cancer therapy, and the use of genetically-modified T-lymphocytes expressing chimeric immunoreceptors in the treatment of human brain tumors and other cancers.

### BACKGROUND OF THE INVENTION

Primary brain tumors are the third leading contributor to cancer-related mortality in young adults, are the second leading contributor in children, and appear to be increasing in incidence both in the pediatric and geriatric population¹⁻⁴. Gliomas are the most common type of primary brain tumors; 20,000 cases are diagnosed and 14,000 glioma-related deaths occur annually in the United States⁵⁻⁸. Gliomas are heterogeneous with respect to their malignant behavior and, in their most common and aggressive forms, anaplastic astrocytoma (AA-grade III) and glioblastoma multiforme (GBM-grade IV), are rapidly progressive and nearly uniformly lethal^{9, 10}. Currently available therapeutic modalities have minimal curative potential for these high-grade tumors and often exacerbate the already severe morbidities imposed by their location in the central nervous system. Thus patients with malignant glioma are often struck in the most productive period of their lives; frequent deterioration of mental faculties and a high case:fatality ratio contribute to the unique personal and social impact of these tumors.

The cornerstones of oncologic management of malignant glioma are resection and radiation therapy¹¹⁻¹⁶. With modern surgical and radiotherapeutic techniques the mean duration of survival has increased to 82 weeks for glioblastoma multiforme and 275 weeks for anaplastic astrocytoma, although 5-year survival rates have only increased from 3 to 6% for glioblastoma multiforme and 12.1% for anaplastic astrocytoma⁶⁻⁸. The major prognostic indicators for prolonged survival are younger age (<40yrs) and performance status (KPS score >70)¹⁷. Resections of >90% of bulky tumors are usually attempted provided that vital functional anatomy is spared. When used in conjunction with post-operative radiation therapy, the impact of extent of resection on duration of survival is less clear^{18, 19}. The addition of chemotherapy to resection and radiation provides only marginal survival advantage to patients with anaplastic astrocytoma or glioblastoma multiforme²⁰⁻²³. Nitrosureas alone or in combination with procarbazine and vincristine are the conventional drugs used in the community and appear to improve the 1-year and 2-year survival rates by 15% without impacting on the overall median survival^{24; 25}. More aggressive regimens incorporating platinum-based drugs and topoisomerase inhibitors are under investigation²⁶. The role of high-dose chemotherapy with stem cell rescue has not been substantiated to date²⁷⁻²⁹.

Approximately 80% of recurrent tumors arise from radiographically enhancing remnants of the original incompletely resected tumor^{10, 30; 31}. Provided recurrences are unifocal and amenable in their location to aggressive re-resection, this approach can extend survival duration, particularly for patients with anaplastic astrocytoma and those glioblastoma multiforme patients with a KPS >70.¹⁰ The median survival of recurrent glioblastoma multiforme patients treated with re-resection is 36 weeks^{10, 30, 31}. Radiation therapy in the form of either brachytherapy or stereotactic radiosurgery may extend the duration of survival in re-resected recurrent glioblastoma multiforme patients by only 10-12 weeks³². The use of chemotherapy in the setting of recurrent disease should be in the context of available clinical trials, as its efficacy in this patient population is unsubstantiated.

The continued dismal prognosis of malignant glioma has prompted the clinical investigation of novel therapeutic entities, including, but not limited to: gene therapy (TK-suicide, antisense inhibition of tumor growth factor receptors, conditionally lethal viral vectors), immunotherapy (antibody, tumor cell vaccines, immunotoxins, adoptive transfer of activated lymphocytes), and anti-angiogenesis approaches³³⁻⁴⁰. The multiplicity of challenges faced in the development of effective adjuvant therapies for malignant glioma include the extensive infiltrative growth of tumor cells into normal brain parenchyma, the capacity of soluble factors elaborated from these tumors to attenuate the development of immune responses, and the difficulty of establishing clinically meaningful therapeutic ratios when administering therapeutics into the central nervous system (CNS). Early clinical evaluation of novel therapeutics is clearly indicated in this patient population.

Recently, receptors for transferring and growth factors have been the subject of experimental glioma therapeutics utilizing ligands for these receptors conjugated to toxins or radionucleotides as a delivery system⁴¹. The specificity of this approach relies on the unique expression or over-expression of targeted receptors on glioma cells compared to normal brain. Interestingly, some receptor complexes for interleukins utilized by the immune system are expressed by gliomas, in particular high-affinity IL-13 receptors⁴²⁻⁴⁸. Unlike the IL-13 receptor trimolecular complex utilized by the immune system, which consists of the IL-13Rα1, the IL-4Rβ, and γc, glioma cells overexpress a unique IL-13Rα2 chain capable of binding IL-13 independently of the requirement for IL-4Rβ or γc^{44, 49, 50}. Like its homologue IL-4, IL-13 has pleotrophic immunoregulatory activity outside the CNS⁵¹⁻⁵³. Both cytokines stimulate IgE production by B lymphocytes and suppress pro-inflammatory cytokine production by macrophages. The immunobiology of IL-13 within the CNS is largely unknown.

Detailed studies by Debinski et al. using autoradiography with radiolabeled IL-13 have demonstrated abundant IL-13 binding on nearly all malignant glioma tissues studied^{42, 45, 46, 48}. Moreover, the binding is highly homogeneous within tumor sections and from single cell analysis^{46, 48}. Scatchard analyses of IL-13 binding to human glioma cell lines reveals on average 17,000-28,000 binding sites/cell⁴⁵. Molecular analysis using probes specific for IL-13Rα2 mRNA fail to demonstrate expression of the glioma-specific receptor by normal brain elements in all CNS anatomic locations^{42, 43}. Furthermore, autoradiography with radiolabeled IL-13 failed to demonstrate detectable specific IL-13 binding in the CNS, suggesting that the shared IL13Rα1/IL-4β/γc receptor is also not expressed at detectable levels in the CNS⁴⁶. These findings were independently verified using immunohistochemical techniques on non-pathologic brain sections with antibodies specific for IL-13Rα1 and IL-4β⁵⁴. Thus IL-13Rα2 stands as the most specific and ubiquitously expressed cell-surface target for glioma described to date.

As a strategy to exploit the glioma-specific expression of IL-13Rα2 in the CNS, molecular constructs of the IL-13 cytokine have been described that fuse various cytotoxins (Pseudomonas exotoxin and Diptheria toxin) to its carboxyl terminal⁵⁵⁻⁵⁸. Internalization of these toxins upon binding to IL-13 receptors is the basis of the selective toxicity of these fusion proteins. These toxins display potent cytotoxicity towards glioma cells in vitro at picomolar concentrations⁵⁵. Human intracranial glioma xenografts in immunodeficient mice can be eliminated by intratumor injection of the IL-13-toxin fusion protein without observed toxicities⁵⁵. These studies support the initiation of clinical investigation utilizing IL-13-directed immunotoxins loco-regionally for malignant glioma.

However, the binding of IL-13-based cytotoxins to the broadly expressed IL-13Rα1/IL-4β/γc receptor complex has the potential of mediating untoward toxicities to normal tissues outside the CNS, and thus limits the systemic administration of these agents. IL-13 has been extensively dissected at the molecular level: structural domains of this cytokine that are important for associating with individual receptor subunits have been mapped^{55, 58}. Consequently, selected amino acid substitutions in IL-13 have predictable effects on the association of this cytokine with its receptor subunits. Amino acid substitutions in IL-13's alpha helix A, in particular at amino acid 13, disrupt its ability to associate with IL-4β, thereby selectively reducing the affinity of IL-13 to the IL-13Rα1/IL-4β/γc receptor by a factor of five^{55, 57, 58}. Surprisingly, binding of mutant IL-13(E13Y) to IL-13Rα2 was not only preserved but increased relative to wild-type IL-13 by 50-fold. Thus, minimally altered IL-13 analogs can simultaneously increase IL-13's specificity and affinity for glioma cells via selective binding to IL-13Rα2 relative to normal tissues bearing IL-13Rα1/IL-4β/γc receptors.

Malignant gliomas represent a clinical entity that is highly attractive for immunotherapeutic intervention since 1) most patients with resection and radiation therapy achieve a state of minimal disease burden and 2) the anatomic location of these tumors within the confines of the CNS make direct loco-regional administration of effector cells possible. At least two pathologic studies have demonstrated that the extent of perivascular lymphocytic infiltration in malignant gliomas correlates with an improved prognosis⁵⁹⁻⁶¹. Animal model systems have established that glioma-specific T cells, but not lymphokine-activated killer (LAK) cells, can mediate the regression of intracerebrally implanted gliomas⁶²⁻⁷¹. T cells, unlike LAK cells, have the capacity to infiltrate into brain parenchyma and thus can target infiltrating tumor cells that may be distant from the primary tumor. Despite these findings, there is a substantial body of evidence that gliomas actively subvert immune destruction, primarily by the elaboration of immunosuppressive cytokines (TGF-β2) and prostaglandins, which, inhibit the induction/amplification of glioma-reactive T cell responses⁷²⁻⁷⁴. These findings have prompted the evaluation of ex vivo expanded anti-glioma effector cells for adoptive therapy as a strategy to overcome tumor-mediated limitations of generating responses in vivo.

At least ten pilot studies involving the administration of ex vivo activated lymphocytes to malignant glioma resection cavities have been reported to date⁷⁵⁻⁸⁵. Despite the variety of effector cell types (LAK, TILs, alloreactive CTLs), their heterogeneous composition/variability of composition from patient to patient, and the often modest in vitro reactivity of these effector cells towards glioma targets, these studies, in aggregate, report an approximate 50% response rate in patients with recurrent/refractory disease with anecdotal long-term survivors. These studies support the premise that a superior clinical effect of cellular immunotherapy for glioma might be expected with homogenous highly potent effector cells.

These pilot studies also report on the safety and tolerability of direct administration of ex vivo activated lymphocytes and interleukin-2 (IL-2), a T cell growth factor, into the resection cavity of patients with malignant glioma^{75; 76, 78, 82, 86-92}. Even at large individual cell doses (>10⁹ cells/dose), as well as high cumulative cell doses (>27×10⁹ cells), toxicities are modest, and typically consist of grade II or less transient headache, nausea, vomiting and fever. As noted above, these studies also employed the co-administration of rhIL-2 to support the in vivo survival of transferred lymphocytes. Multiple doses given either concurrently with lymphocytes or sequentially after lymphocyte administration were tolerated at doses as high as 1.2×10⁶ IU/dose for 12-dose courses of IL-2 delivered every 48-hours.

Based on the findings outlined above, strategies to improve the anti-tumor potency of lymphocyte effector cells used in glioma immunotherapy are under development. One approach utilizes bi-specific antibodies capable of co-localizing and activating T lymphocytes via an anti-CD3 domain with glioma targets utilizing an epidermal growth factor receptor (EGFR) binding domain⁹³⁻⁹⁶. Preliminary clinical experience with this bi-specific antibody in combination with autologous lymphocytes suggests that T cells are activated in situ in the resection cavity. Targeting infiltrating tumor cells within the brain parenchyma, however, is a potentially significant limitation of this approach. T cells might have significantly increased anti-glioma activity if they are specific for target antigens expressed by gliomas. A growing number of human genes encoding tumor antigens to which T lymphocytes are reactive have been cloned, including the SART-1 gene, which appears to be expressed by nearly 75% of high-grade gliomas⁹⁷. Both dendritic cell-based in vitro cell culture techniques, as well as tetramer-based T cell selection technologies are making feasible the isolation of antigen-specific T cells for adoptive therapy. Since antigens like SART-1 are recognized by T cells in the context of restricting HLA alleles, antigen-specific approaches will require substantial expansion in the number of antigens and restricting HLA alleles capable of presenting these antigens to be broadly applicable to the general population of glioma patients.

Chimeric antigen receptors engineered to consist of an extracellular single chain antibody (scFvFc) fused to the intracellular signaling domain of the T cell antigen receptor complex zeta chain (scFvFc:ζ) have the ability, when expressed in T cells, to redirect antigen recognition based on the monoclonal antibody's specificity⁹⁸. The design of scFvFc:ζ receptors with target specificities for tumor cell-surface epitopes is a conceptually attractive strategy to generate antitumor immune effector cells for adoptive therapy as it does not rely on pre-existing anti-tumor immunity. These receptors are “universal” in that they bind antigen in a MHC independent fashion, thus, one receptor construct can be used to treat a population of patients with antigen-positive tumors. Several constructs for targeting human tumors have been described in the literature including receptors with specificities for Her2/Neu, CEA, ERRB-2, CD44v6, and epitopes selectively expressed on renal cell carcinoma⁹⁸⁻¹⁰⁴. These epitopes all share the common characteristic of being cell-surface moieties accessible to scFv binding by the chimeric T cell receptor. In vitro studies have demonstrated that both CD4+ and CD8+ T cell effector functions can be triggered via these receptors. Moreover, animal models have demonstrated the capacity of adoptively transferred scFvFc:ζ expressing T cells to eradicate established tumors¹⁰⁵. The function of primary human T cells expressing tumor-specific scFvFc:ζ receptors have been evaluated in vitro; these cells specifically lyse tumor targets and secrete an array of pro-inflammatory cytokines including IL-2, TNF, IFN-γ, and GM-CSF¹⁰⁴. Phase I pilot adoptive therapy studies are underway utilizing autologous scFvFc:ζ-expressing T cells specific for HIV gp120 in HIV infected individuals and autologous scFvFc:ζ-expressing T cells with specificity for TAG-72 expressed on a variety of adenocarcinomas, including breast and colorectal adenocarcinoma.

Investigators at City of Hope have engineered a CD20-specific scFvFc:ζ receptor construct for the purpose of targeting CD20+ B-cell malignancy and an L1-CAM-specific chimeric immunoreceptor for targeting neuroblastoma¹⁰⁶. Preclinical laboratory studies have demonstrated the feasibility of isolating and expanding from healthy individuals and lymphoma patients CD8+ CTL clones that contain a single copy of unrearranged chromosomally integrated vector DNA and express the CD20-specific scFvFc:ζ receptor¹⁰⁷. To accomplish this, purified linear plasmid DNA containing the chimeric receptor sequence under the transcriptional control of the CMV immediate/early promoter and the NeoR gene under the transcriptional control of the SV40 early promoter was introduced into activated human peripheral blood mononuclear cells by exposure of cells and DNA to a brief electrical current, a procedure called electroporation. Utilizing selection, cloning, and expansion methods currently employed in FDA-approved clinical trials at the Fred Hutchinson Cancer Research Center, Seattle, Wash., gene modified CD8+ CTL clones with CD20-specific cytolytic activity have been generated from each of six healthy volunteers in 15 separate electroporation procedures. These clones when co-cultured with a panel of human CD20+ lymphoma cell lines proliferate, specifically lyse target cells, and are stimulated to produce cytokines.

### SUMMARY OF THE INVENTION

The present invention relates to chimeric transmembrane immunoreceptors, named “zetakines,” comprised of an extracellular domain comprising a soluble receptor ligand linked to a support region capable of tethering the extracellular domain to a cell surface, a transmembrane region and an intracellular signaling domain. Zetakines, when expressed on the surface of T lymphocytes, direct T cell activity to those cells expressing a receptor for which the soluble receptor ligand is specific. Zetakine chimeric immunoreceptors represent a novel extension of antibody-based immunoreceptors for redirecting the antigen specificity of T cells, with application to treatment of a variety of cancers, particularly via the autocrine/paracrine cytokine systems utilized by human malignancy.

In one preferred embodiment exploiting the tumor-restricted expression of IL-13Rα2 by malignant glioma and renal cell carcinoma as a target for cellular immunotherapy, a mutant of the IL-13 cytokine, IL-13(E13Y), having selective high-affinity binding to IL-13Rα2 has been converted into a type I transmembrane chimeric immunoreceptor capable of redirecting T cell antigen specificity to IL-13Rα2-expressing tumor cells. This embodiment of the zetakine consists of extracellular IL-13(E13Y) fused to human IgG4 Fc, transmembrane CD4, and intracellular T cell antigen receptor CD3 complex zeta chain. Analogous immunoreceptors can be created that are specific to any of a variety of cancer cell types that selectively express receptors on their cell surfaces, for which selective ligands are known or can be engineered.

Bulk lines and clones of human T cells stably transformed to express such an immunoreceptor display redirected cytolysis of the cancer cell type to which they are specific, while showing negligible toxicity towards non-target cells. Such engineered T cells are a potent and selective therapy for malignancies, including difficult to treat cancers such as glioma.

### BRIEF DESCRIPTION OF THE FIGURES

FIG. 1: Results of a Western Blot showing that the IL13zetakine Chimeric Immunoreceptor is expressed as an intact glycosylated protein in Jurkat T cells.

FIG. 2: Results of flow cytometric analysis showing that expressed IL13zetakine chimeric immunoreceptor trafficks to the cell-surface as a type I transmembrane protein.

FIG. 3: Results of flow cytometric analysis showing the cell surface phenotype of a representative primary human IL13zetakine⁺ CTL clone.

FIG. 4: Results of a chromium release assays showing (a) that the IL13zetakine⁺ CTL clone acquired glioma-specific re-directed cytolytic activity, and (b) the profile of anti-glioma cytolytic activity by primary human IL13zetakine⁺ CD8⁺ CTL clones was observed in glioma cells generally.

FIG. 5: Results of in vitro stimulation of cytokine production, showing that IL13zetakine⁺ CTL clones are activated for cytokine production by glioma stimulator cells.

FIG. 6: Results of in vitro stimulation of cytokine production, showing the specific inhibition of IL13zetakine⁺ CTL activation for cytokine production by anti-IL13R Mab and rhIL13.

FIG. 7: Results of growth studies showing (a) that IL13zetakine⁺ CD8^{+ CTL cells proliferate upon co-culture with glioma stimulators, and (b) the inhibition of glioma-stimulated proliferation of IL}13zetakine⁺ CD8^{+ CTL cells by rhIL-}13.

FIG. 8: Flow chart of the construction of IL13zetakine/HyTK-pMG.

FIG. 9: Plasmid map of IL13zetakine/HyTK-pMG.

### DETAILED DESCRIPTION

An ideal cell-surface epitope for tumor targeting with genetically-engineered re-directed T cells would be expressed solely on tumor cells in a homogeneous fashion and on all tumors within a population of patients with the same diagnosis. Modulation and/or shedding of the target molecule from the tumor cell membrane may also impact on the utility of a particular target epitope for re-directed T cell recognition. To date few “ideal” tumor-specific epitopes have been defined and secondary epitopes have been targeted based on either lack of expression on critical normal tissues or relative over-expression on tumors. In the case of malignant glioma, the intracavitary administration of T cells for the treatment of this cancer permits the expansion of target epitopes to those expressed on tumor cells but not normal CNS with less stringency on expression by other tissues outside the CNS. The concern regarding toxicity from cross-reactivity of tissues outside the CNS is mitigated by a) the sequestration of cells in the CNS based on the intracavitary route of administration and b) the low cell numbers administered in comparison to cell doses typically administered systemically.

The IL-13Rα2 receptor stands out as the most ubiquitous and specific cell-surface target for malignant glioma⁴⁷. Sensitive autoradiographic and immunohistochemical studies fail to detect IL-13 receptors in the CNS^{46, 48}. Moreover, mutation of the IL-13 cytokine to selectively bind the glioma-restricted IL-13Rα2 receptor is a further safeguard against untoward reactivity of IL-13-directed therapeutics against IL-13Rα1/IL-4β+ normal tissues outside the CNS^{55, 57}. The potential utility of targeting glioma IL-13Rα2 the design and testing of a novel engineered chimeric immunoreceptor for re-directing the specificity of T cells that consists of an extracellular IL-13 mutant cytokine (E13Y) tethered to the plasma membrane by human IgG4 Fc which, in turn, is fused to CD4TM and the cytoplasmic tail of CD3 zeta. This chimeric immunoreceptor has been given the designation of “IL-13 zetakine”. The IL-13Rα2 receptor/IL-13(E13Y) receptor-ligand pair is an excellent guide for understanding and assessing the suitability of receptor-ligand pairs generally for use in zetakines. An ideal zetakine comprises an extracellular soluble receptor ligand having the properties of IL-13(E13Y) (specificity for a unique cancer cell surface receptor, in vivo stability due to it being derived from a naturally-occurring soluble cell signal molecule, low immunogenicity for the same reason). The use of soluble receptor ligands as distinct advantages over the prior art use of antibody fragments (such as the scFvFc immunoreceptors) or cell adhesion molecules, in that soluble receptor ligands are more likely to be stable in the extracellular environment, non-antigenic, and more selective.

Chimeric immunoreceptors according to the present invention comprise an extracellular domain comprised of a soluble receptor ligand linked to an extracellular support region that tethers the ligand to the cell surface via a transmembrane domain, in turn linked to an intracellular receptor signaling domain. Examples of suitable soluble receptor ligands include autocrine and paracrine growth factors, chemokines, cytokines, hormones, and engineered artificial small molecule ligands that exhibit the required specificity. Natural ligand sequences can also be engineered to increase their specificity for a particular target cell. Selection of a soluble receptor ligand for use in a particular zetakine is governed by the nature of the target cell, and the qualities discussed above with regard to the IL-13(E13Y) molecule, a preferred ligand for use against glioma. Examples of suitable support regions include the constant (Fc) regions of immunoglobins, human CD8∝, and artificial linkers that serve to move the targeting moiety away from the cell surface for improved access to receptor binding on target cells. A preferred support region is the Fc region of an IgG (such as IgG4). Examples of suitable transmembrane domains include the transmembrane domains of the leukocyte CD markers, preferably that of CD8. Examples of intracellular receptor signaling domains are those of the T cell antigen receptor complex, preferably the zeta chain of CD3 also Fcγ RIII costimulatory signaling domains, CD28, DAP10, CD2, alone or in a series with CD3zeta.

In the IL-13 zetakine embodiment, the human IL-13 cDNA having the El13Y amino acid substitution was synthesized by PCR splice overlap extension. A full length IL-13 zetakine construct was assembled by PCR splice overlap extension and consists of the human GM-CSF receptor alpha chain leader peptide, IL-13(E13Y)-Gly-Gly-Gly, human IgG4 Fc, human CD4TM, and human cytoplasmic zeta chain. This cDNA construct was ligated into the multiple cloning site of a modified pMG plasmid under the transcriptional control of the human Elongation Factor-1alpha promoter (Invivogen, San Diego). This expression vector co-expresses the HyTK cDNA encoding the fusion protein HyTK that combines in a single molecule hygromycin phosphotransferase activity for in vitro selection of transfectants and HSV thymidine kinase activity for in vivo ablation of cells with ganciclovir from the CMV immediate/early promoter. Western blot of whole cell Jurkat lysates pre-incubated with tunicamycin, an inhibitor of glycosylation, with an anti-zeta antibody probe demonstrated that the expected intact 56-kDa chimeric receptor protein is expressed. This receptor is heavily glycosylated consistent with post-translational modification of the native IL-13 cytokine¹⁰⁸. Flow cytometric analysis of IL-13 zetakine⁺ Jurkat cells with anti-human IL-13 and anti-human Fc specific antibodies confirmed the cell-surface expression of the IL-13 zetakine as a type I transmembrane protein.

Using established human T cell genetic modification methods developed at City of Hope¹⁰⁷, primary human T cell clones expressing the IL-13 zetakine chimeric immunoreceptor have been generated for pre-clinical functional characterization. IL-13 zetakine+ CD8+ CTL clones display robust proliferative activity in ex vivo expansion cultures. Expanded clones display re-directed cytolytic activity in 4-hr chromium release assays against human IL-13Rα2+ glioblastoma cell lines. The level of cytolytic activity correlates with levels of zetakine expression on T cells and IL-13Rα2 receptor density on glioma target cells. In addition to killing, IL-13 zetakine+ clones are activated for cytokine secretion (IFN-γ, TNF-α, GM-CSF). Activation was specifically mediated by the interaction of the IL-13 zetakine with the IL-13Rα2 receptor on glioma cells since CTL clones expressing an irrelevant chimeric immunoreceptor do not respond to glioma cells, and, since activation can be inhibited in a dose-dependent manner by the addition to culture of soluble IL-13 or blocking antibodies against IL-13 on T cell transfectants and IL-13Rα2 on glioma target cells. Lastly, IL-13 zetakine-expressing CD8+CTL clones proliferate when stimulated by glioma cells in culture. IL-13 zetakine+CTL clones having potent anti-glioma effector activity will have significant clinical activity against malignant gliomas with limited collateral damage to normal CNS.

An immunoreceptor according to the present invention can be produced by any means known in the art, though preferably it is produced using recombinant DNA techniques. A nucleic acid sequence encoding the several regions of the chimeric receptor can prepared and assembled into a complete coding sequence by standard techniques of molecular cloning (genomic library screening, PCR, primer-assisted ligation, site-directed mutagenesis, etc.) . The resulting coding region is preferably inserted into an expression vector and used to transform a suitable expression host cell line, preferably a T lymphocyte cell line, and most preferably an autologous T lymphocyte cell line. A third party derived T cell line/clone, a transformed humor or xerogenic immunologic effector cell line, for expression of the immunoreceptor. NK cells, macrophages, neutrophils, LAK cells, LIK cells, and stem cells that differentiate into these cells, can also be used. In a preferred embodiment, lymphocytes are obtained from a patient by leukopharesis, and the autologous T cells are transduced to express the zetakine and administered back to the patient by any clinically acceptable means, to achieve anti-cancer therapy.

Suitable doses for a therapeutic effect would be between about 10⁶ and about 10⁹ cells per dose, preferably in a series of dosing cycles. A preferred dosing regimen consists of four one-week dosing cycles of escalating doses, starting at about 10⁷ cells on Day 0, increasing incrementally up to a target dose of about 10⁸ cells by Day 5. Suitable modes of administration include intravenous, subcutaneous, intracavitary (for example by reservoir-access device), intraperitoneal, and direct injection into a tumor mass.

The following examples are solely for the purpose of illustrating one embodiment of the invention.

### EXAMPLE 1

Construction of an Immunoreceptor Coding Sequence

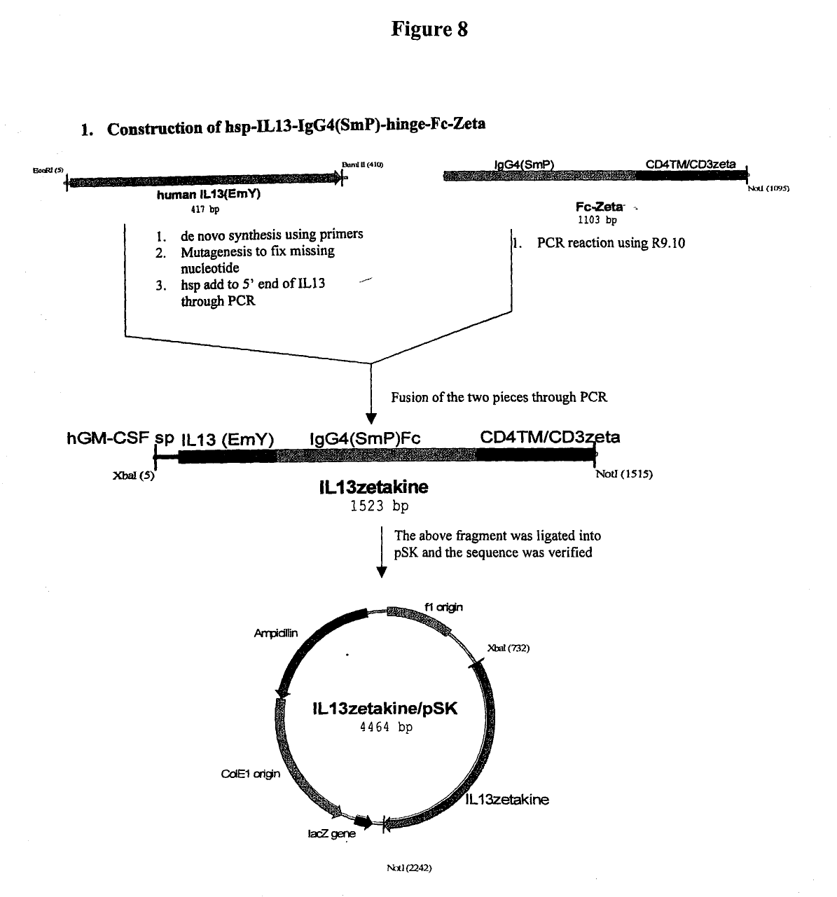

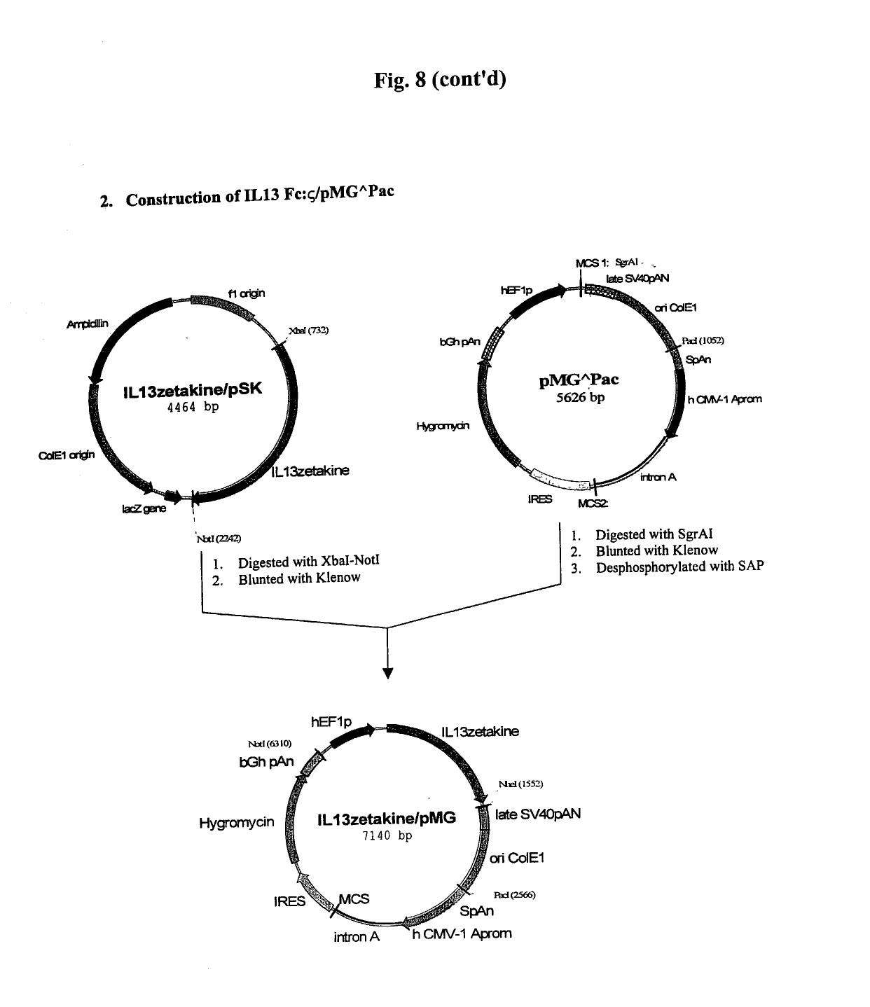

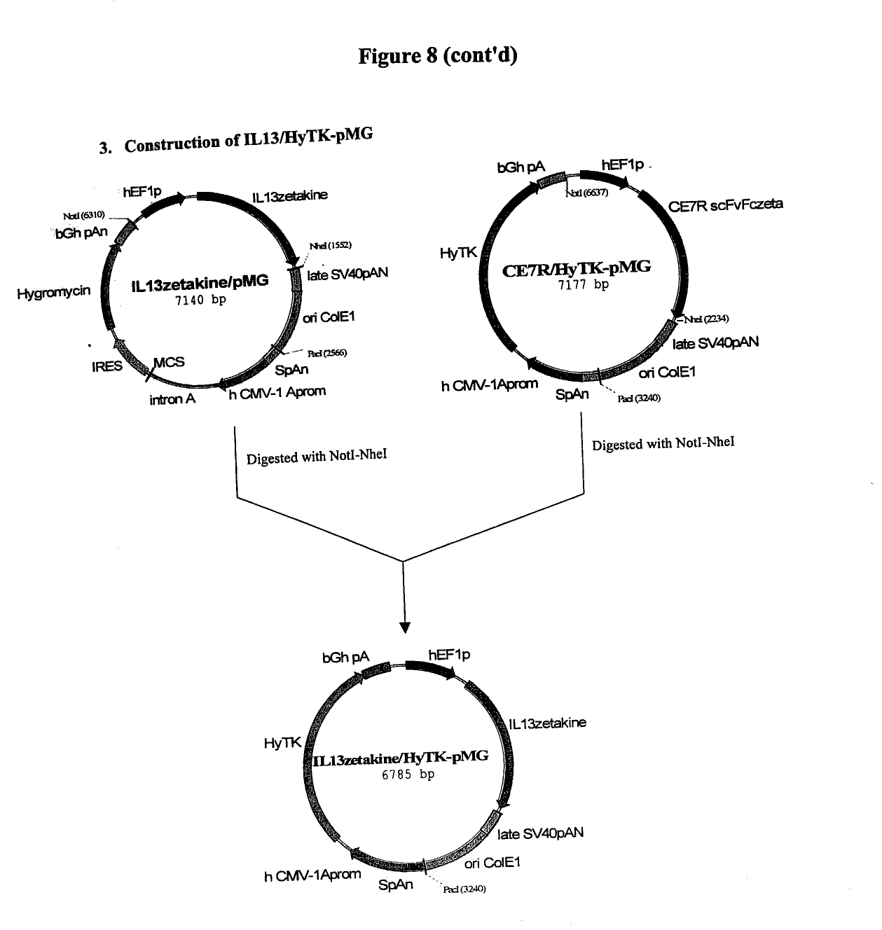

The coding sequence for an immunoreceptor according to the present invention was constructed by de novo synthesis of the IL13(E13Y) coding sequence using the following primers (see FIG. 8 for a flow chart showing the construction of the immunoreceptor coding sequence and expression vector):

IL13P1:

| | |
|---|---|
| EcoRI | [SEQ ID NO.1] |

TATGAATTCATGGCGCTTTTGTTGACCACGGTCATTGCTCTCACTTGCC
TTGGCGGCTTTGCCTCCCCAGGCCCTGTGCCTCCCTCTACAGCCCTCAG
GTAC
1L13P2:

[SEQ ID NO.2]

GTTGATGCTCCATACCATGCTGCCATTGCAGAGCGGAGCCTTCTGGTTC
TGGGTGATGTTGACCAGCTCCTCAATGAGGTACCTGAGGGCTGTAGAG
GGAG
1L13P3:

[SEQ ID NO.3]

CTCTGGGTCTTCTCGATGGCACTGCAGCCTGACACGTTGATCAGGGATT
CCAGGGCTGCACAGTACATGCCAGCTGTCAGGTTGATGCTCCATACCAT
GC
1L13P4:

[SEQ ID NO.4]

CCTCGATTTTGGTGTCTCGGACATGCAAGCTGGAAAACTGCCCAGCTGA
GACCTTGTGCGGGCAGAATCCGCTCAGCATCCTCTGGGTCTTCTCGATG
GC
1L13P5:

| | |
|---|---|
| BamHI | [SEQ ID NO.5] |

TCGGATCCTCAGTTGAACCGTCCCTCGCGAAAAAGTTTCTTTAAATGTA
AGAGCAGGTCCTTTACAAACTGGGCCACCTCGATTTTGGTGTCTCGG

The final sequence (417bp) was end-digested with EcoRI-BamHI, and ligated into the plasmid pSK (stratagene, LaJolla, Calif.) as ligation 312#3. Ligation 312#3 was mutagenized (stratagene kit, per manufacturer's instructions) to fix a deleted nucleotide using the primers 5′: IL13 312#3 mut5-3 (CAACCTGACAGCTGGCATGTACTGTGCAGCCCTGGAATC [SEQ ID NO. 6]) and 3′:IL13 312#3 mut3-5 (GATTCCAGGGCTGCACAGTACATGCCAGCTGTCAGGTTG [SEQ ID NO. 7]), and ligation 312#3 as a template, to form ligation 348#1 (IL13zetakine/pSK).

The coding Human GM-CSFR alpha chain Signal Peptide (hsp) coding sequence was fused to the 5′ end of IL13(E13Y) by standard PCR splice overlap extension. The hsp sequence (101 bp) was obtained from the template ligation 301#10 (hsp/pSK) (human GCSF receptor α-chain leader sequence from human T cell cDNA), using the primers 5′:19hsp5′(ATCTCTAGAGCCGCCACCATGCTTCTCCTGGTGACAAGCCTTC [SEQ ID NO. 8]) (XbaI site highlighted in bold), and 3′: hsp-IL13FR (GAGGGAGGCACAGGGCCTGGGATCAGGAGGAATG [SEQ ID NO. 9]). The IL-13 sequence (371 bp) was obtained using the primers 5′: hsp-IL13FF (CATTCCTCCTGATCCCAGGCCCTGTGCCTCCCTC [SEQ ID NO. 10]) and 3′: IL13-IgG4FR (GGGACCATATTTGGACTCGTTGAACCGTCCCTCGC [SEQ ID NO. 11]), and ligation 312#3 as template. Fusion was achieved using the 101 bp hsp sequence and 371 bp IL13 sequence thus obtained, and the primers 5′: 19hsp5′ and 3′: IL13-IgG4FR, to yield a 438 bp fusion hsp-IL13 sequence.

A sequence encoding the IgG4 Fc region IgG4m:zeta was fused to the 3′ end of the hsp-IL13 fusion sequence using the same methods. The IgG4m:zeta sequence (1119 bp) was obtained using the primers 5′: IL13-IgG4FF (GCGAGGGACGGTTCAACGAGTCCAAATATGGTCCC [SEQ ID NO. 12]) and 3′: ZetaN3′ (ATGCGGCCGCTCAGCGAGGGGGCAGG [SEQ ID NO. 13]) (NotI site highlighted in bold), using the sequence R9.10 (IgG4mZeta/pSK) as template. The 1119 bp IgG4m:zeta sequence was fused to the hsp-IL13 fusion sequence using the respective sequences as templates, and the primers 5′: 19hsp5′ and 3′: ZetaN3′, to yield a 1522 bp hsp-IL13-IgG4m:zeta fusion sequence. The ends were digested with XbaI-NotI, and ligated into pSK as ligation 351#7, to create the plasmid IL13zetakine/pSK (4464 bp).

### EXAMPLE 2

Construction of Expression Vector

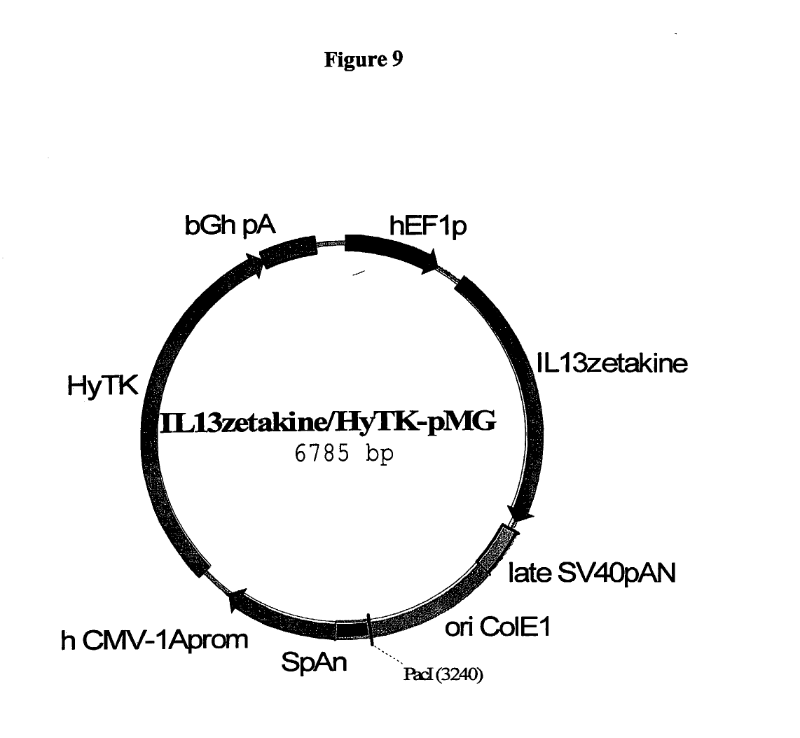

An expression vector containing the IL13 zetakine coding sequence was created by digesting the IL13zetakine/pSK of Example 1 with XbaI-NotI, and creating blunt ends with Klenow, ,and ligating the resulting fragment into the plasmid pMG\hat{ }Pac (Invirogen) (first prepared by opening with SgrAI, blunting with Klenow, and dephosphorylation with SAP), to yield the plasmid IL13zetakine/pMG. See FIG. 8. The hygromycin resistance region of IL13zetakine/pMG was removed by digestion with NotI-NheI, and replaced by the selection/suicide fusion HyTK, obtained from plasmid CE7R/HyTK-pMG (Jensen, City of Hope) by digestion with NotI-NheI, to create the expression vector IL13zetakine/HyTK-pMG (6785 bp). This plasmid comprises the Human Elongation Factor-1α promoter (hEF1p) at bases 6-549, the IL13zetakine coding sequence at bases 692-2185, the Simian Virus 40 Late polyadenylation signal (Late SV40pAN) at bases 2232-2500, a minimal E. coli origin of replication (Ori ColE1) at bases 2501-3247, a synthetic poly A and Pause site (SpAN) at bases 3248-3434, the Immeate-early CMV enhancer/promoter (h CMV-1Aprom) at bases 3455-4077, the Hygromycin resistance-Thymidine kinase coding region fusion (HyTK) at bases 4259-6334, and the bovine growth hormone polyadenylation signal and a transcription pause (BGh pAn) at bases 6335-6633. The plasmid has a PacI linearization site at bases 3235-3242. The hEF1p and IL13zetakine elements derived from IL13zetakine/pMG, and the remaining elements derived from CE7R/HyTk-pMG (and with the exception of the HyTK element, ultimately from the parent plasmid pMG\hat{ }Pac). In sum, IL13zetakine/HyTK-pMG is a modified pMG backbone, expressing the IL13zetakine gene from the hEF1promoter, and the HyTK fusion from the h CMV-1A promoter. A map of the plasmid IL13zetakine/HyTK-pMG appears in FIG. 9.

### EXAMPLE 3

Expression of the Immunoreceptor

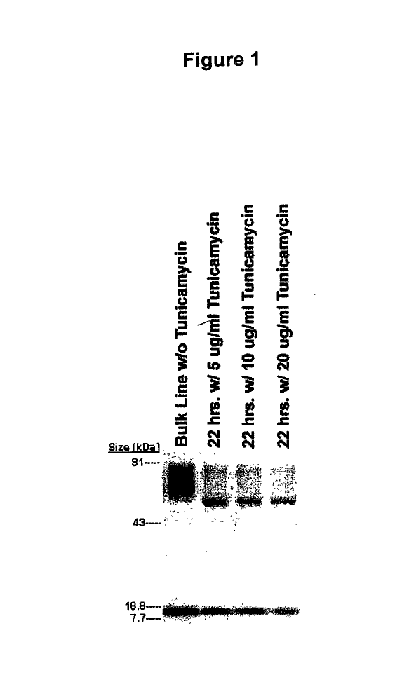

Assessment of the integrity of the expressed construct was first delineated by Wester blot probed with an anit-zeta antibody of whole cell lysates derived from Jurkat T cell stable transfectants¹⁰⁷ cocultured in the presence or absence of tunicamycin, an inhibitor of glycosylation. FIG. 1. Jurkat T cell stable transfectants (Jurkat-IL13-pMG bulk line) were obtained by electroporating Jurkat T cells with the IL13zetakine/HyTK-pMG expression vector, followed by selection and expansion of positive transfectants. 2×10⁶ cells from the Jurkat-IL13-pMG bulk line were plated per well in a 24-well plate with or without 5 μg/ml, 10 μg/ml, or 20 μg/ml Tunicamycin. The plate was incubated at 37° C. for 22 hrs. Cells were harvested from each well, and each sample was washed with PBS and resuspended in 50 μl RIPA buffer (PBS, 1% NP40, 0.5% sodium deoxycholate, 0.1% SDS) containing 1 tablet/10 ml Complete Protease Inhibitor Cocktail (Boehringer Mannheim, Indianapolis, Ind.). Samples were incubated on ice for 30 minutes then disrupted by aspiration with syringe with 21 gauge needle then incubated on ice for an additional 30 minutes before being centrifuged at 4° C. for 20 minutes at 14,000 rpm. Samples of centrifuged lysate supernatant were harvested and boiled in an equal volume of sample buffer under reducing conditions, then subjected to SDS-PAGE electrophoresis on a 12% acrylamide gel. Following transfer to nitrocellulose, membrane was allowed to dry O/N at 4° C. Next morning, membrane was blocked in a Blotto solution containing 0.04 gm/ml non-fat dried milk in T-TBS (0.02% Tween 20 in Tris buffered saline pH 8.0) for 1 hour. Membrane was then incubated with primary mouse anti-human CD3ζ monoclonal antibody (Pharmingen, San Diego, Calif.) at a concentration of 1 μg/ml for 2 hours, washed, and then incubated with a 1:3000 dilution (in Blotto solution) of goat anti-mouse IgG alkaline phosphatase conjugated secondary antibody (Bio-Rad ImmunoStar Kit, Hercules, Calif.) for 1 hour. Prior to developing, membrane was washed 4 additional times in T-TBS, and then incubated with 3 ml of phosphatase substrate solution (Biorad ImmunoStar Kit, Hercules, Calif.) for 5 minutes at room temperature. Membrane was then covered with plastic, and exposed to x-ray film. Consistant with the known glycosylation pattern of wild-type human IL-13, the electrophoretic mobility of expressed IL-13(E13Y) zetakine is demonstrative of a heavily glycosylated protein which, when expressed in the presence of tunicamycin, is reduced to an amino acid backbone of approximately 54 kDa.

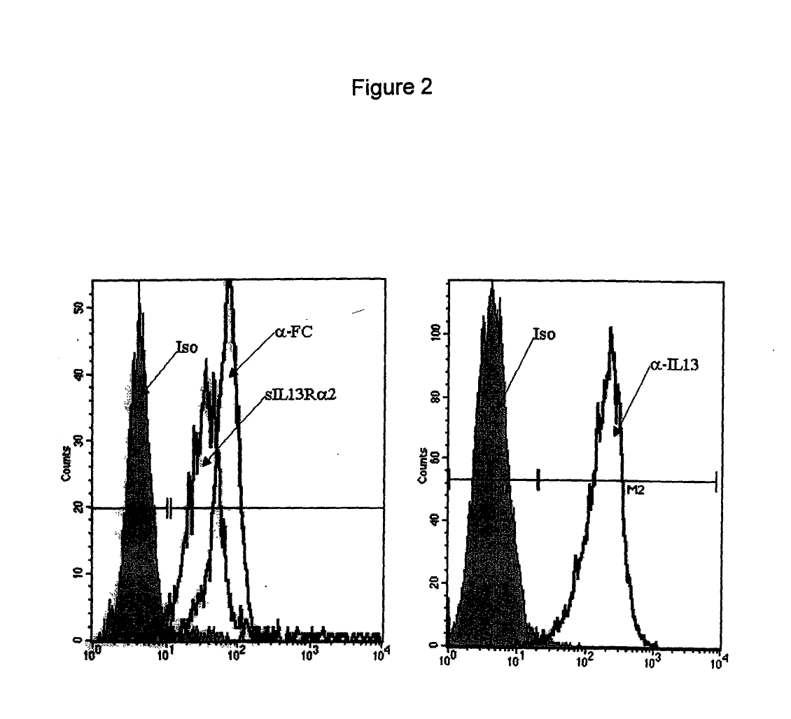

The IL-13(E13Y) zetakine traffics to the cell surface as a homodimeric type I transmembrane protein, as evidenced by flow cytometric analysis of transfectants with a phycoerythrin (PE)-conjugated anti human-IL13 monoclonal antibody and a fluorescein isothiocyanate (FITC)-conjugated mouse anti-human Fc (gamma) fragment-specific F(ab′)₂ antibody. FIG. 2. Jurkat IL13zetakine-pMG transfectants were stained with anti-human Fc(FITC) antibody (Jackson ImmunoResearch, West Grove, Pa.), recombinant human IL13Rα2/human IgG1chimera (R&D Systems, Minneapolis, Minn.) followed by FITC-conjugated anti human-IgG1monoclonal antibody (Sigma, St. Louis, Mo.), and an anti-IL13(PE) antibody (Becton Dickinson, San Jose, Calif.) for analysis of cell surface chimeric receptor expression. Healthy donor primary cells were also stained with FITC-conjugated anti-CD4, anti-CD8, anti-TCR, and isotype control monoclonal antibodies (Becton Dickinson, San Jose, Calif.) to assess cell surface phenotype. For each stain, 10⁶ cells were washed and resuspended in 100 μl of PBS containing 2% FCS, 0.2 mg/ml NaN₃, and 5 μl of stock antibody. Following a 30 minute incubation at 4° C., cells were washed twice and either stained with a secondary antibody, or resuspended in PBS containing 1% paraformaldehyde and analyzed on a FACSCaliber cytometer. EXAMPLE 4

Binding of IL13(E13Y) Zetakine to IL13Rα2 Receptor

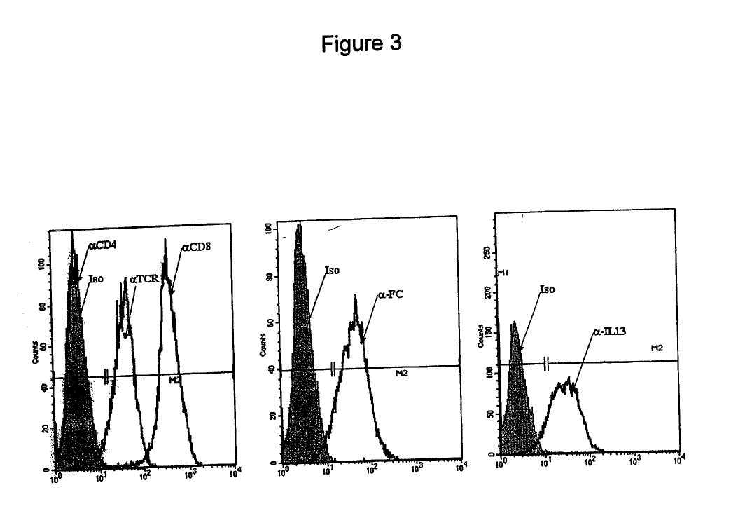

IL-13(E13Y), tethered to the cell membrane by human IgG4 Fc (i.e., IL13(E13Y) zetakine), is capable of binding to its target IL13Rα2 receptor as assessed by flow cytometric analysis using soluble IL13Rα2-Fc fusion protein. FIG. 3. Cloned human PBMC IL13zetakine-pMG transfectants were obtained by electroporating PBMC with the IL13zetakine/HyTK-pMG expression vector, followed by selection and expansion of positive transfectants¹⁰⁷. IL13zetakine⁺ CTL clonal cells were stained with a fluorescein isothiocyanate (FITC)-conjugated mouse anti-human Fc (gamma) fragment-specific F(ab′)₂ (Jackson ImmunoResearch, West Grove, Pa.), recombinant human IL13Rα2/human IgG1 chimera (R&D Systems, Minneapolis, Minn.) followed by FITC-conjugated anti human-IgG1 monoclonal antibody (Sigma, St. Louis, Mo.), and a phycoerythrin (PE)-conjugated anti human-IL13 monoclonal antibody (Becton Dickinson, San Jose, Calif.) for analysis of cell surface chimeric receptor expression. Healthy donor primary cells were also stained with FITC-conjugated anti-CD4, anti-CD8, anti-TCR, and isotype control monoclonal antibodies (Becton Dickinson, San Jose, Calif.) to assess cell surface phenotype. For each stain, 10⁶ cells were washed and resuspended in 100 μl of PBS containing 2% FCS, 0.2 mg/ml NaN₃, and 5 μl of antibody. Following a 30 minute incubation at 4° C., cells were washed twice and either stained with a secondary antibody, or resuspended in PBS containing 1% paraformaldehyde and analyzed on a FACSCaliber cytometer.

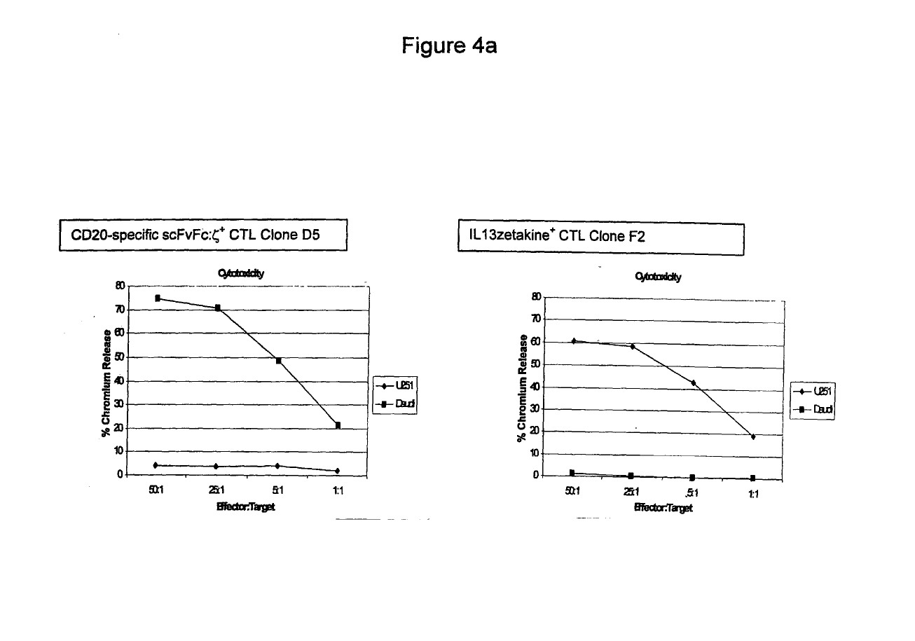

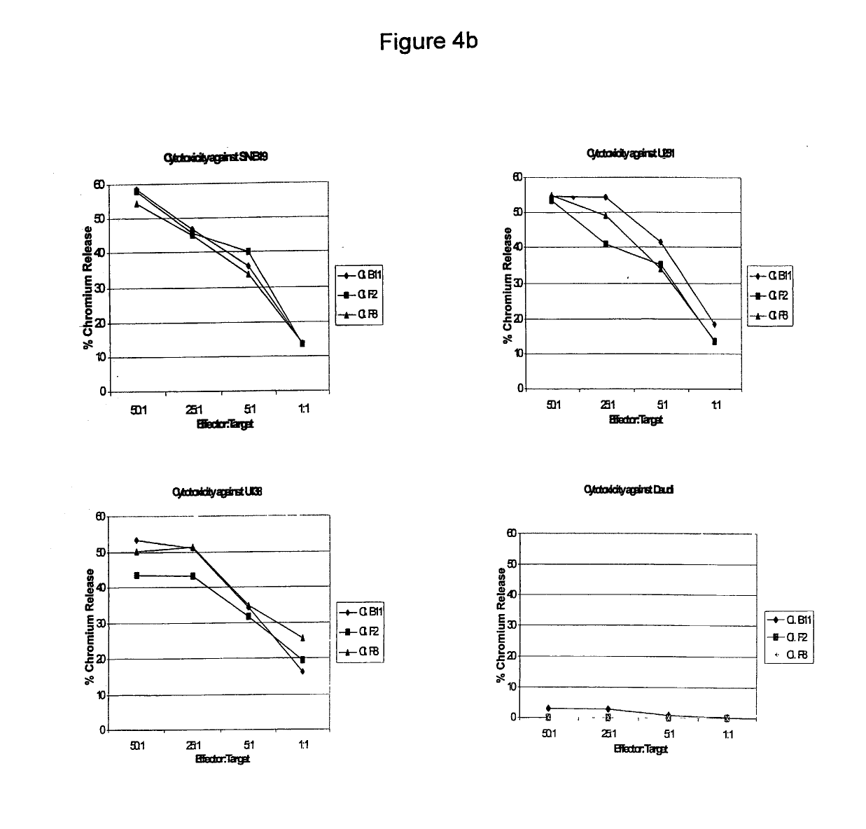

Next, the immunobiology of the IL-13(E13Y) zetakine as a surrogate antigen receptor for primary human T cells was evaluated. Primary human T cells were electroporated with the plasmid expression vector. Positive transformants were selected with hygromycin, cloned in limiting dilution, then expanded by recursive stimulation cyles with OKT3, IL-2 and irradiated feeder cells. Clones demonstrating IL 13zetakine expression by Western blot and FACS were then subjected to functional evaluation in 4-hr chromium release assays against a variety of IL-13α2⁺/CD20³¹ glioma cell lines (U251, SN-B19, U138), and the IL-13α⁻/CD20⁺ B cell lyphocyte line Daudi). These tests showed that IL13zetakine conferred cytolytic activity that was specific for glioma cells (FIG. 4a), and that this specific cytolytic activity is present for glioma cells as a class (FIG. 4b). The cytolytic activity of MJ-IL13-pMG clones was assayed by employing ⁵¹Cr-labeled SN-B19, U251, and U138 glioma cell lines (IL13α2+/CD20−) and Daudi (CD20+/IL13α2−) as targets. MJ-IL13 effectors were assayed 8-12 days following stimulation. Effectors were harvested, washed, and resuspeded in assay media: 2.5×10⁵, 1.25×10⁵, 2.5×10⁴, and 5×10³ effectors were cultured in triplicate at for 4 hours with 5×10³ target cells in 96-well V-bottom microtiter plates. After incubation, 100 μl aliquots of cell-free supernatant were harvested and ⁵¹Cr in the supernatants was assayed with a γ-counter. Percent specific cytolysis was calculated as follows:

$$\frac{( Experimental {}^{51} Cr release ) - ( control {}^{51} Cr release )}{( Maximum {}^{51} Cr release ) - ( control {}^{51} Cr release )} \times 100$$

Control wells contained target cells incubated in the presence of target cells alone. Maximum ⁵¹Cr release was determined by measuring the ⁵¹Cr released by labeled target cells in the presence of 2% SDS. Bulk lines of stabley transfected human T cells consisting of approximately 40% IL-13(E13Y) zetakine⁺ TCRα/β⁺ lymphocytes displayed re-directed cytolysis specific for 13Rα2⁺ glioma targets in 4-hr chromium release assays (>50% specific lysis at E:T ratios of 25:1), with negligable acitivity against IL-13Rα2³¹ targets (<8% specific lysis at E:T ratios of 25:1). IL-13(E13Y) zetakine⁺CD8⁺TCRα/β⁺ CTL clones selected on the basis of high-level binding to anti-IL-13 antibody also display redirected IL13Rα2−specific glioma cell killing. FIG. 4b.

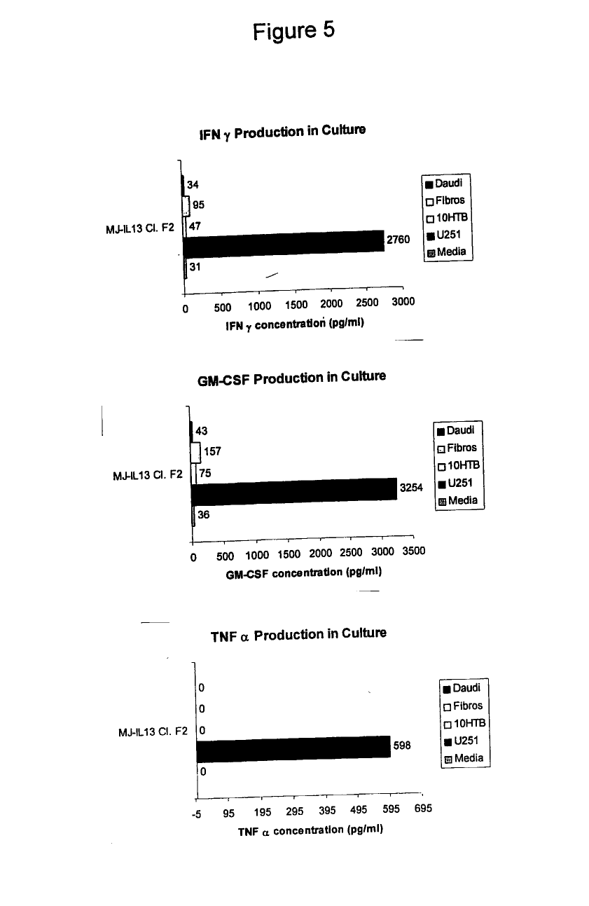

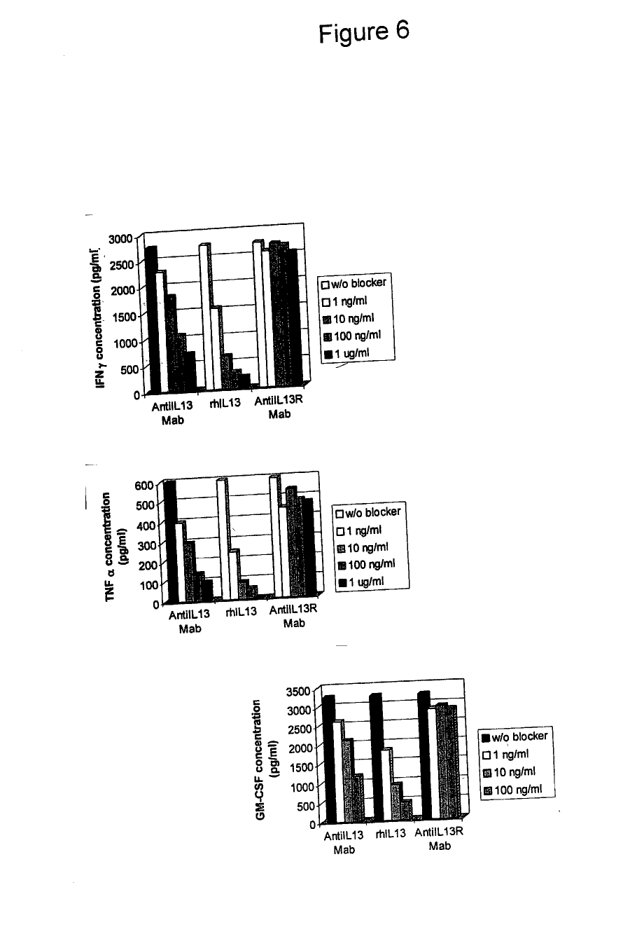

IL-13 zetakine-expressing CD8⁺ CTL clones are activated and proliferate when stimulated by glioma cells in culture. FIGS. 5-7. MJ-IL13-pMG C1. F2 responder cells expressing the IL13 zetakine were evaluated for receptor-mediated triggering of IFNγ, GM-CSF, and TNFα production in vitro. 2×10⁶ responder cells were co-cultured in 24-well tissue culture plates with 2×10⁵ irradiated stimulator cells (Daudi, Fibroblasts, Neuroblastoma 10HTB, and glioblastoma U251) in 2 ml total. Blocking rat anti-human-IL13 monoclonal antibody (Pharmingen, San Diego, Calif.), recombinant human IL13 (R&D Systems, Minneapolis, Minn.), and IL13Rα2−specific goat IgG (R&D Systems, Minneapolis, Minn.) were added to aliquots of U251 stimulator cells (2×10⁵/ml) at concentrations of 1 ng/ml, 10 ng/ml, 100 ng/ml, and 1 μg/ml, 30 minutes prior to the addition of responder cells. Plates were incubated for 72 hours at 37° C., after which time culture supernatants were harvested, aliquoted, and stored at −70° C. ELISA assays for IFNγ, GM-CSF, and TNFα were carried out using the R&D Systems (Minneapolis, Minn.) kit per manufacturer's instructions. Samples were tested in duplicate wells undiluted or diluted at 1:5 or 1:10. The developed ELISA plate was evaluated on a microplate reader and cytokine concentrations determined by extrapolation from a standard curve. Results are reported as picograms/ml, and show strong activation for cytokine production by glioma stimulator cells. FIG. 5, FIG. 6.

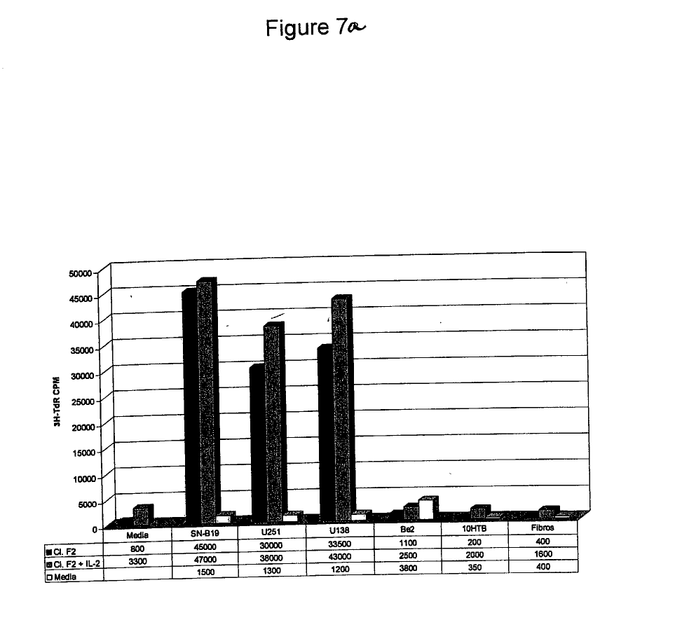

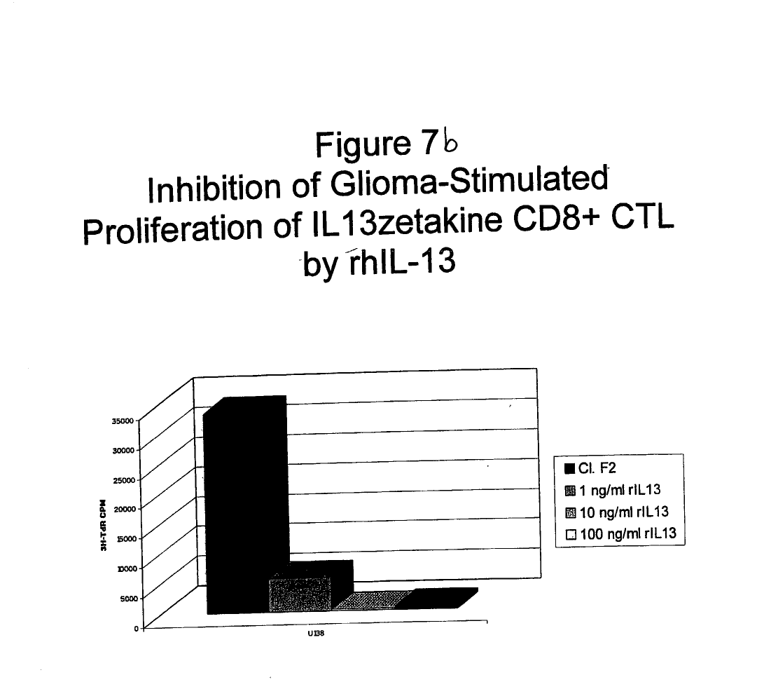

Lastly, IL-2 independent proliferation of IL13zetakine⁺ CD8⁺ CTL was observed upon co-cultivation with glioma stimulators (FIG. 7a), but not with IL13 Rα2 stimulators. Proliferation was inhibited by the addition of rhIL-13 antibody (FIG. 7b), showing that the observed proliferation was dependant on binding of zetakine to the IL-13Rα2 glioma cell-specific receptor.

### EXAMPLE 5

Preparation of IL-13 zetakine⁺ T Cells Suitable for Therapeutic Use

The mononuclear cells are separated from heparinized whole blood by centrifugation over clinical grade Ficoll (Pharmacia, Uppsula, Sweden). PBMC are washed twice in sterile phosphate buffered saline (Irvine Scientific) and suspended in culture media consisting of RPMI 1640 HEPES, 10% heat inactivated FCS, and 4 mM L-glutamine. T cells present in patient PBMC are polyclonally activated by addition to culture of Orthoclone OKT3 (30 ng/ml). Cell cultures are then incubated in vented T75 tissue culture flasks in the study subject's designated incubator. Twenty-four hours after initiation of culture rhIL-2 is added at 25 U/ml.

Three days after the initiation of culture PBMC are harvested, centrifuged, and resuspended in hypotonic electroporation buffer (Eppendort) at 20×10⁶ cells/ml. 25 μg of the plasmid IL13zetakine/HyTK-pMG of Example 3, together with 400 μl of cell suspension, are added to a sterile 0.2 cm electroporation cuvette. Each cuvette is subjected to a single electrical pulse of 250V/40 μs and again incubated for ten minutes at RT. Surviving cells are harvested from cuvettes, pooled, and resuspended in culture media containing 25 U/ml rhIL-2. Flasks are placed in the patient's designated tissue culture incubator. Three days following electroporation hygromycin is added to cells at a final concentration of 0.2 mg/ml. Electroporated PBMC are cultured for a total of 14 days with media and IL-2 supplementation every 48-hours.

The cloning of hygromycin-resistant CD8+ CTL from electroporated OKT3-activated patient PBMC is initiated on day 14 of culture. Briefly, viable patient PBMC are added to a mixture of 100×10⁶ cyropreserved irradiated feeder PBMC and 20×10⁶ irradiated TM-LCL in a volume of 200 ml of culture media containing 30 ng/ml OKT3 and 50 U/ml rhIL-2. This mastermix is plated into ten 96-well cloning plates with each well receiving 0.2 ml. Plates are wrapped in aluminum foil to decrease evaporative loss and placed in the patient's designated tissue culture incubator. On day 19 of culture each well receives hygromycin for a final concentration of 0.2 mg/ml. Wells are inspected for cellular outgrowth by visualization on an inverted microscope at Day 30 and positive wells are marked for restimulation.

The contents of each cloning well with cell growth are individually transferred to T25 flasks containing 50×10⁶ irradiated PBMC, 10×10⁶ irradiated LCL, and 30 ng/mlOKT3 in 25 mls of tissue culture media. On days 1,3,5,7,9,11, and 13 after restimulation flasks receive 50 U/ml rhIL-2 and 15 mls of fresh media. On day 5 of the stimulation cycle flasks are also supplemented with hygromycin 0.2 mg/ml. Fourteen days after seeding cells are harvested, counted, and restimulated in T75 flasks containing 150×10⁶ irradiated PBMC, 30×10⁶ irradiated TM-LCL and 30 ng/ml OKT3 in 50 mls of tissue culture media. Flasks receive additions to culture of rhIL-2 and hygromycin as outlined above.

CTL selected for expansion for possible use in therapy are analyzed by immunofluorescence on a FACSCalibur housed in CRB-3006 using FITC-conjugated monoclonal antibodies WT/31 (aβTCR), Leu 2a (CD8), and OKT4 (CD4) to confirm the requisite phenotype of clones (αβTCR+, CD4−, CD8+, and IL13+). Criteria for selection of clones for clinical use include uniform TCR αβ+, CD4−, CD8+and IL13+ as compared to isotype control FITC/PE-conjugated antibody. A single site of plasmid vector chromosomal integration is confirmed by Southern blot analysis. DNA from genetically modified T cell clones will be screened with a DNA probe specific for the plasmid vector. Probe DNA specific for the HyTK in the plasmid vector is synthesized by random priming with florescein-conjugated dUTP per the manufacture's instructions (Amersham, Arlington Hts, Ill.). T cell genomic DNA is isolated per standard technique. Ten micrograms of genomic DNA from T cell clones is digested overnight at 37° C. then electrophoretically separated on a 0.85% agarose gel. DNA is then transferred to nylon filters (BioRad, Hercules, Calif.) using an alkaline capillary transfer method. Filters are hybridized overnight with probe in 0.5 M Na₂PO₄, pH 7.2, 7% SDS, containing 10 μg/ml salmon sperm DNA (Sigma) at 65° C. Filters are then washed four times in 40 mM Na₂PO₄, pH 7.2, 1% SDS at 65° C. and then visualized using a chemiluminescence AP-conjugated anti-florescein antibody (Amersham, Arlington Hts, Ill.). Criteria for clone selection is a single band unique vector band.

Expression of the IL-13 zetakine is determined by Western blot procedure in which chimeric receptor protein is detected with an anti-zeta antibody. Whole cell lysates of transfected T cell clones are generated by lysis of 2×10⁷ washed cells in 1 ml of RIPA buffer (PBS, 1% NP40, 0.5% sodium deoxycholate, 0.1% SDS) containing 1 tablet/10 ml Complete Protease Inhibitor Cocktail (Boehringer Mannheim). After an eighty minute incubation on ice, aliquots of centrifuged whole cell lysate supernatant are harvested and boiled in an equal volume of loading buffer under reducing conditions then subjected to SDS-PAGE electrophoresis on a precast 12% acrylamide gel (BioRad). Following transfer to nitrocellulose, membranes are blocked in blotto solution containing 0.07 gm/ml non-fat dried milk for 2 hours. Membranes are washed in T-TBS (0.05% Tween 20 in Tris buffered saline pH 8.0) then incubated with primary mouse anti-human CD3ζ monoclonal antibody 8D3 (Pharmingen, San Diego, Calif.) at a concentration of 1 μg/ml for 2 hours. Following an additional four washes in T-TBS, membranes are incubated with a 1:500 dilution of goat anti-mouse IgG alkaline phosphatase-conjugated secondary antibody for 1 hour. Prior to developing, membranes are rinsed in T-TBS then developed with 30 ml of “AKP” solution (Promega, Madison, Wis.) per the manufacturer's instructions. Criteria for clone selection is the presence of a chimeric zeta band.

CD8+ cytotoxic T cell clones expressing the IL-13 zetakine chimeric immunoreceptor recognize and lyse human glioblastoma target cells following interaction of the chimeric receptor with the cell surface target epitope in a HLA-unrestricted fashion. The requirements for target IL-13Rα2 epitope expression and class I MHC independent recognition will be confirmed by assaying each aβTCR+, CD8+, CD4−, IL-13 zetakine+CTL clones against IL-13Rα2+ Daudi cell transfectants and IL-13Rα2-Daudi cells. T cell effectors are assayed 12-14 days following stimulation with OKT3. Effectors are harvested, washed, and resuspended in assay media; and Daudi cell transfectants expressing IL-13Rα2. 2.5×10⁵, 1.25×10⁵, 0.25×10⁵, and 0.05×10⁵ effectors are plated in triplicate at 37° C. for 4 hours with 5×10³ target cells in V-bottom microtiter plates (Costar, Cambridge, Mass.). After centrifugation and incubation, 100 μL aliquots of cell-free supernatant is harvested and counted. Percent specific cytolysis is calculated as follows:

$$\frac{( Experimental {}^{51} Cr release ) - ( control {}^{51} Cr release )}{( Maximum {}^{51} Cr release ) - ( control {}^{51} Cr release )} \times 100$$

Control wells contain target cells incubated in assay media. Maximum ⁵¹Cr release is determined by measuring the ⁵¹Cr content of target cells lysed with 2% SDS. Criteria for clone selection is >25% specific lysis of IL-13Rα2+ Daudi transfectants at an E:T ratio of 5:1 and a <10% lysis of parental Daudi at the same E:T ratio.

### EXAMPLE 6

Treatment of Human Glioma Using IL-13 Zetakine-expressing T Cells.

T cell clones genetically modified according to Example 5 to express the IL-13R zetakine chimeric immunoreceptor and HyTK are selected for:

a. TCRα/β+, CD4⁻, CD8⁺, IL-13⁺cell surface phenotype as determined by flow cytometry.

b. Presence of a single copy of chromosomally integrated plasmid vector DNA as evidenced by Southern blot.

C. Expression of the IL-13 zetakine protein as detected by Western blot.

d. Specific lysis of human IL-13Rα2⁺ targets in 4-hr chromium release assays.

e. Dependence on exogenous IL-2 for in vitro growth.

f. Mycoplasma, fungal, bacterial sterility and endotoxin levels <5 EU/ml.

g. In vitro sensitivity of clones to ganciclovir.

Peripheral blood mononuclear cells are obtained from the patient by leukapheresis, preferably following recovery from initial resection surgery and at a time at least three weeks from tapering off steroids and/or their most recent systemic chemotherapy. The target leukapheresis mononuclear cell yield is 5×10⁹ and the target number of hygromycin-resistant cytolytic T cell clones is 25 with the expectation that at least five clones will be identified that meet all quality control parameters for ex-vivo expansion. Clones are cryopreserved and patients monitored by serial radiographic and clinical examinations. When recurrence of progression of disease is documented, patients undergo a re-resection and/or placement of a reservoir-access device (Omaya reservoir) for delivering T cells to the tumor resection cavity. Following recovery from surgery and tapering of steroids, if applicable, the patient commences with T cell therapy.

The patient receives a target of at least four one-week cycles of therapy. During the first cycle, cell dose escalation proceeds from an initial dose on Day 0 of 10⁷ cells, followed by 5×10⁷ cells on Day 3 to the target dose of 10⁸ cells on Day 5. Cycle 2 commences as early as one week from commencement of cycle 1. Those patients demonstrating tumor regression with residual disease on MRI may have additional courses of therapy beginning no earlier than Week 7 consisting of repetition of Cycles 3 and 4 followed by one week of rest/restaging provided these treatments are well tolerated (max. toxicities <grade 3) until such time that disease progression or a CR is achieved based on radiographic evaluation.

Cell doses are at least a log less than doses given in studies employing intracavitary LAK cells (individual cell doses of up to 10⁹ and cumulative cell numbers as high as 2.75×10¹⁰ have been safety administered), ex vivo expanded TILs (up to 10⁹ cells/dose reported with minimal toxicity) and allo-reactive lymphocyte (starting cell dose 10⁸ with cumulative cell doses up to 51.5×10⁸) delivered to a similar patient population⁷⁵⁻⁸⁵. The rationale for the lower cell doses as proposed in this protocol is based on the increased in vitro reactivity/anti-tumor potency of IL-13 zetakine+ CTL clones compared to the modest reactivity profile of previously utilized effector cell populations. Low-dose repetitive dosing is favored to avoid potentially dangerous inflammatory responses that might occur with single large cell number instillations. Each infusion will consist of a single T cell clone. The same clone will be administered throughout a patient's treatment course. On the days of T cell administration, expanded clones are aseptically processed by washing twice in 50 cc of PBS then resuspended in pharmaceutical preservative-free normal saline in a volume that results in the cell dose for patient delivery in 2 mls. T cells are instilled over 5-10 minutes. A 2 ml PFNS flush will be administered over 5 minutes following T cells. Response to therapy is assessed by brain MRI +/− gandolinium, with spectroscopy.

Expected side-effects of administration of T cells into glioma resection cavities typically consist of self-limited nausea and vomiting, fever, and transient worsening of existing neurological deficits. These toxicities can be attributed to both the local inflammation/edema in the tumor bed mediated by T cells in combination with the action of secreted cytokines. These side-effects typically are transient and less than grade II in severity. Should patients experience more severe toxicities it is expected that decadron alone or in combination with ganciclovir will attenuate the inflammatory process and ablate the infused cells. The inadvertent infusion of a cell product that is contaminated with bacteria or fungus has the potential of mediating serious or life-threatening toxicities. Extensive pre-infusion culturing of the cell product is conducted to identify contaminated tissue culture flasks and minimize this possibility. On the day of re-infusion, gram stains of culture fluids, as well as, endotoxin levels are performed.

Extensive molecular analysis for expression of IL-13Rα2 has demonstrated that this molecule is tumor-specific in the context of the CNS^{44, 46, 48, 54}. Furthermore, the only human tissue with demonstrable IL-13Rα2 expression appears to be the testis⁴². This tumor-testis restrictive pattern of expression is reminiscent of the growing number of tumor antigens (i.e. MAGE, BAGE, GAGE) expressed by a variety of human cancers, most notably melanoma and renal cell carcinoma¹⁰⁹⁻¹¹¹. Clinical experience with vaccine and adoptive T cell therapy has demonstrated that this class of antigens can be exploited for systemic tumor immunotherapy without concurrent autoimmune attack of the testis¹¹²⁻¹¹⁴. Presumably this selectively reflects the effect of an intact blood-testis barrier and an immunologically privileged environment within the testis. Despite the exquisite specificity of the mutant IL-13 targeting moiety, toxicities are theoretically possible if cells egress into the systemic circulation in sufficient numbers and recognize tissues expressing the IL-13Rα1/IL-4β receptor. In light of this remote risk, as well as the possibility that instilled T cells in some patients may mediate an overly exuberant inflammatory response in the tumor bed, clones are equipped with the HyTK gene which renders T cells susceptible to in vivo ablation with ganciclovir¹¹⁵⁻¹¹⁸. Ganciclovir-suicide, in combination with an intra-patient T cell dose escalation strategy, helps minimize the potential risk to research participants.

Side effects associated with therapy (headache, fever, chills, nausea, etc.) are managed using established treatments appropriate for the condition. The patient receives ganciclovir if any new grade 3 or any grade 4 treatment-related toxicity is observed that, in the opinion of the treating physician, puts that patient at significant medical danger. Parentally administered ganciclovir is dosed at 10 mg/kg/day divided every 12 hours. A 14-day course will be prescribed but may be extended should symptomatic resolution not be achieved in that time interval. Treatment with ganciclovir leads to the ablation of IL-13 zetakine⁺ HyTK⁺ CD8⁺ CTL clones. Patients should be hospitalized for the first 72 hours of ganciclovir therapy for monitoring purposes. If symptoms do not respond to ganciclovir within 48 hours additional immunosuppressive agents including but not limited to corticosteroids and cyclosporin may be added at the discretion of the treating physician. If toxicities are severe, decadron and/or other immunosuppressive drugs along with ganciclovir are used earlier at the discretion of the treating physician.

### REFERENCES

1. Davis F G, McCarthy B J. Epidemiology of brain tumors. Curr Opin Neurol. 2000;13:635-640.

2. Davis F G, Malinski N, Haenszel W, et al. Primary brain tumor incidence rates in four United States regions, 1985-1989: a pilot study. Neuroepidemiology. 1996;15:103-112.

3. Smith M A, Freidlin B, Ries L A, Simon R. Increased incidence rates but no space-time clustering of childhood astrocytoma in Sweden, 1973-1992: a population-based study of pediatric brain tumors. Cancer. 2000;88:1492-1493.

4. Ahsan H, Neugut A I, Bruce J N. Trends in incidence of primary malignant brain tumors in USA, 1981-1990. Int J Epidemiol. 1995;24:1078-1085.

5. Ashby L S, Obbens E A, Shapiro W R. Brain tumors. Cancer Chemother Biol Response Modif. 1999;18:498-549.

6. Davis F G, Freels S, Grutsch J, Barlas S, Brem S. Survival rates in patients with primary malignant brain tumors stratified by patient age and tumor histological type: an analysis based on Surveillance, Epidemiology, and End Results (SEER) data, 1973-1991. J Neurosurg. 1998;88:1-10.

7. Duffner P K, Cohen M E, Myers M H, Heise H W. Survival of children with brain tumors: SEER Program, 1973-1980. Neurology. 1986;36:597-601.

8. Davis F G, Freels S, Grutsch J, Barlas S, Brem S. Survival rates in patients with primary malignant brain tumors stratified by patient age and tumor histological type: an analysis based on Surveillance, Epidemiology, and End Results (SEER) data, 1973-1991. J Neurosurg. 1998;88: 1.-10.

9. Kolles H, Niedermayer I, Feiden W. [Grading of astrocytomas and oligodendrogliomas]. Pathologe. 1998; 19:259-268.

10. Huncharek M, Muscat J. Treatment of recurrent high grade astrocytoma; results of a systematic review of 1,415 patients. Anticancer Res. 1998; 18:1303-1311.

11. Loiseau H, Kantor G. [The role of surgery in the treatment of glial tumors]. Cancer Radiother. 2000;4 Suppl 1:48s-52s.

12. Palma L. Trends in surgical management of astrocytomas and other brain gliomas. Forum (Genova). 1998;8:272-281.

13. Azizi S A, Miyamoto C. Principles of treatment of malignant gliomas in adults: an overview. J Neurovirol. 1998;4:204-216.

14. Shapiro W R, Shapiro J R. Biology and treatment of malignant glioma. Oncology (Huntingt). 1998; 12:233-240.

15. Chamberlain M C, Kormanik P A. Practical guidelines for the treatment of malignant gliomas. West J Med. 1998;168:114-120.

16. Ushio Y. Treatment of gliomas in adults. Curr Opin Oncol. 1991;3:467-475.

17. Scott J N, Rewcastle N B, Brasher P M, et al. Long-term glioblastoma multiforme survivors: a population-based study. Can J Neurol Sci. 1998;25:197-201.

18. Finlay J L, Wisoff J H. The impact of extent of resection in the management of malignant gliomas of childhood. Childs Nerv Syst. 1999; 15:786-788.

19. Hess K R. Extent of resection as a prognostic variable in the treatment of gliomas. J Neurooncol. 1999;42:227-231.

20. van den Bent M J. Chemotherapy in adult malignant glioma. Front Radiat Ther Oncol. 1999;33:174-191.

21. DeAngelis L M, Burger P C, Green S B, Cairncross J G. Malignant glioma: who benefits from adjuvant chemotherapy? Ann Neurol. 1998;44:691-695.

22. Armstrong T S, Gilbert M R. Chemotherapy of astrocytomas: an overview. Semin Oncol Nurs. 1998;14:18-25.

23. Prados M D, Russo C. Chemotherapy of brain tumors. Semin Surg Oncol. 1998;14:88-95.

24. Prados M D, Scott C, Curran W J, Nelson D F, Leibel S, Kramer S. Procarbazine, lomustine, and vincristine (PCV) chemotherapy for anaplastic astrocytoma: A retrospective review of radiation therapy oncology group protocols comparing survival with carmnustine or PCV adjuvant chemotherapy. J Clin Oncol. 1999;17:3389-3395.

25. Fine H A, Dear K B, Loeffler J S, Black P M, Canellos G P. Meta-analysis of radiation therapy with and without adjuvant chemotherapy for malignant gliomas in adults. Cancer. 1993;71:2585-2597.

26. Mahaley M S, Gillespie G Y. New therapeutic approaches to treatment of malignant gliomas: chemotherapy and immunotherapy. Clin Neurosurg. 1983;31:456-469.

27. Millot F, Delval O, Giraud C, et al. High-dose chemotherapy with hematopoietic stem cell transplantation in adults with bone marrow relapse of medulloblastoma: report of two cases. Bone Marrow Transplant. 1999;24:1347-1349.

28. Kalifa C, Valteau D, Pizer B, Vassal G, Grill J, Hartmann O. High-dose chemotherapy in childhood brain tumours. Childs Nerv Syst. 1999; 15:498-505.

29. Finlay J L. The role of high-dose chemotherapy and stem cell rescue in the treatment of malignant brain tumors. Bone Marrow Transplant. 1996; 18 Suppl 3:S1-S5.

30. Brandes A A, Vastola F, Monfardini S. Reoperation in recurrent high-grade gliomas: literature review of prognostic factors and outcome. Am J Clin Oncol. 1999;22:387-390.

31. Miyagi K, Ingram M, Techy G B, Jacques D B, Freshwater D B, Sheldon H. Immunohistochemical detection and correlation between MHC antigen and cell-mediated immune system in recurrent glioma by APAAP method. Neurol Med Chir (Tokyo). 1990;30:649-655.

32. Bauman G S, Sneed P K, Wara W M, et al. Reirradiation of primary CNS tumors. Int J Radiat Oncol Biol Phys. 1996;36:433-441.

33. Fine H A. Novel biologic therapies for malignant gliomas. Antiangiogenesis, immunotherapy, and gene therapy. Neurol Clin. 1995;13:827-846.

34. Brandes A A, Pasetto L M. New therapeutic agents in the treatment of recurrent high-grade gliomas. Forum (Genova). 2000;10:121-131.

35. Pollack I F, Okada H, Chambers W H. Exploitation of immune mechanisms in the treatment of central nervous system cancer. Semin Pediatr Neurol. 2000;7:131-143.

36. Black K L, Pikul B K. Gliomas—past, present, and future. Clin Neurosurg. 1999;45:160-163.

37. Riva P, Franceschi G, Arista A, et al. Local application of radiolabeled monoclonal antibodies in the treatment of high grade malignant gliomas: a six-year clinical experience. Cancer. 1997;80:2733-2742.

38. Liang B C, Weil M. Locoregional approaches to therapy with gliomas as the paradigm. Curr Opin Oncol. 1998;10:201-206.

39. Yu J S, Wei M X, Chiocca E A, Martuza R L, Tepper R I. Treatment of glioma by engineered interleukin 4-secreting cells. Cancer Res. 1993;53:3125-3128.

40. Alavi J B, Eck S L. Gene therapy for malignant gliomas. Hematol Oncol Clin North Am. 1998;12:617-629.

41. Debinski W. Recombinant cytotoxins specific for cancer cells. Ann N Y Acad Sci. 1999;886:297-299.

42. Debinski W, Gibo D M. Molecular expression analysis of restrictive receptor for interleukin 13, a brain tumor-associated cancer/testis antigen. Mol Med. 2000;6:440-449.

43. Mintz A, Debinski W. Cancer genetics/epigenetics and the X chromosome: possible new links for malignant glioma pathogenesis and immune-based therapies. Crit Rev Oncog. 2000;1 1:77-95.

44. Joshi B H, Plautz G E, Puri R K. Interleukin-13 receptor alpha chain: a novel tumor-associated transmembrane protein in primary explants of human malignant gliomas. Cancer Res. 2000;60:1168-1172.

45. Debinski W, Obiri N I, Powers S K, Pastan I, Puri R K. Human glioma cells over express receptors for interleukin 13 and are extremely sensitive to a novel chimeric protein composed of interleukin 13 and pseudomonas exotoxin. Clin Cancer Res. 1995;1:1253-1258.

46. Debinski W, Gibo D M, Hulet S W, Connor J R, Gillespie G Y. Receptor for interleukin 13 is a marker and therapeutic target for human high-grade gliomas. Clin Cancer Res. 1999;5:985-990.

47. Debinski W. An immune regulatory cytokine receptor and glioblastoma multiforme: an unexpected link. Crit Rev Oncog. 1998;9:255-268.

48. Debinski W, Slagle B, Gibo D M, Powers S K, Gillespie G Y. Expression of a restrictive receptor for interleukin 13 is associated with glial transformation. J Neurooncol. 2000;48:103-111.

49. Debinski W, Miner R, Leland P, Obiri N I, Puri R K. Receptor for interleukin (IL) 13 does not interact with IL4 but receptor for IL4 interacts with IL13 on human glioma cells. J Biol Chem. 1996;271 :22428-22433.

50. Murata T, Obiri N I, Debinski W, Puri R K. Structure of IL-13 receptor: analysis of subunit composition in cancer and immune cells. Biochem Biophys Res Commun. 1997;238:90-94.

51. Opal S M, DePalo V A. Anti-inflammatory cytokines. Chest. 2000;117:1162-1172.

52. Romagnani S. T-cell subsets (Th1 versus Th2). Ann Allergy Asthma Immunol. 2000;85:9-18.

53. Spellberg B, Edwards J E, Jr. Type 1/Type 2 immunity in infectious diseases. Clin Infect Dis. 2001;32:76-102.

54. Liu H, Jacobs B S, Liu J, et al. Interleukin-13 sensitivity and receptor phenotypes of human glial cell lines: non-neoplastic glia and low-grade astrocytoma differ from malignant glioma. Cancer Immunol Immunother. 2000;49:319-324.

55. Debinski W, Gibo D M, Obiri N I, Kealiher A, Puri R K. Novel anti-brain tumor cytotoxins specific for cancer cells. Nat Biotechnol. 1998;16:449-453.

56. Debinski W, Gibo D M, Puri R K. Novel way to increase targeting specificity to a human glioblastoma-associated receptor for interleukin 13. Int J Cancer. 1998;76:547-551.

57. Debinski W, Thompson J P. Retargeting interleukin 13 for radioimmunodetection and radioimmunotherapy of human high-grade gliomas. Clin Cancer Res. 1999;5:3143s-3147s.

58. Thompson J P, Debinski W. Mutants of interleukin 13 with altered reactivity toward interleukin 13 receptors. J Biol Chem. 1999;274:29944-29950.

59. Brooks W H, Netsky M G, Levine J E. Immunity and tumors of the nervous system. Surg Neurol. 1975;3:184-186.

60. Bullard D E, Gillespie G Y, Mahaley M S, Bigner D D. Immunobiology of human gliomas. Semin Oncol. 1986;13:94-109.

61. Coakham H B. Immunology of human brain tumors. Eur J Cancer Clin Oncol. 1984;20:145-149.

62. Holladay F P, Heitz T, Wood G W. Antitumor activity against established intracerebral gliomas exhibited by cytotoxic T lymphocytes, but not by lymphokine-activated killer cells. J Neurosurg. 1992;77:757-762.

63. Holladay F P, Heitz T, Chen Y L, Chiga M, Wood G W. Successful treatment of a malignant rat glioma with cytotoxic T lymphocytes. Neurosurgery. 1992;31:528-533.

64. Kruse C A, Lillehei K O, Mitchell D H, Kleinschmidt-DeMasters B, Bellgrau D. Analysis of interleukin 2 and various effector cell populations in adoptive immunotherapy of 9L rat gliosarcoma: allogeneic cytotoxic T lymphocytes prevent tumor take. Proc Natl Acad Sci USA. 1990;87:9577-9581.

65. Miyatake S, Nishihara K, Kikuchi H, et al. Efficient tumor suppression by glioma-specific murine cytotoxic T lymphocytes transfected with interferon-gamma gene. J Natl Cancer Inst. 1990;82:217-220.

66. Plautz G E, Touhalisky J E, Shu S. Treatment of murine gliomas by adoptive transfer of ex vivo activated tumor-draining lymph node cells. Cell Immunol. 1997;178:101-107.

67. Saris S C, Spiess P, Lieberman D M, Lin S, Walbridge S, Oldfield E H. Treatment of murine primary brain tumors with systemic interleukin-2 and tumor-infiltrating lymphocytes. J Neurosurg. 1992;76:513-519.

68. Tzeng J J, Barth R F, Clendenon N R, Gordon W A. Adoptive immunotherapy of a rat glioma using lymphokine-activated killer cells and interleukin 2. Cancer Res. 1990;50:4338-4343.

69. Yamasaki T, Kikuchi H. An experimental approach to specific adoptive immunotherapy for malignant brain tumors. Nippon Geka Hokan. 1989;58:485-492.

70. Yamasaki T, Handa H, Yamashita J. Watanabe Y, Namba Y, Hanaoka M. Specific adoptive immunotherapy with tumor-specific cytotoxic T-lymphocyte clone for murine malignant gliomas. Cancer Res. 1984;44:1776-1783.

71. Yamasaki T, Handa H, Yamashita J, Watanabe Y, Namba Y, Hanaoka M. Specific adoptive immunotherapy of malignant glioma with long-term cytotoxic T lymphocyte line expanded in T-cell growth factor. Experimental study and future prospects. Neurosurg Rev. 1984;7:37-54.

72. Kikuchi K, Neuwelt E A. Presence of immunosuppressive factors in brain-tumor cyst fluid. J Neurosurg. 1983;59:790-799.

73. Yamanaka R, Tanaka R, Yoshida S, Saitoh T, Fujita K, Naganuma H. Suppression of TGF-beta1 in human gliomas by retroviral gene transfection enhances susceptibility to LAK cells. J Neurooncol. 1999;43:27-34.

74. Kuppner M C, Hamou M F, Bodmer S, Fontana A, de Tribolet N. The glioblastoma-derived T-cell suppressor factor/transforming growth factor beta 2 inhibits the generation of lymphokine-activated killer (LAK) cells. Int J Cancer. 1988;42:562-567.

75. Hayes R L. The cellular immunotherapy of primary brain tumors. Rev Neurol (Paris). 1992;148:454-466.

76. Ingram M, Buckwalter J G, Jacques D B, et al. Immunotherapy for recurrent malignant glioma: an interim report on survival. Neurol Res. 1990;12:265-273.

77. Jaeckle K A. Immunotherapy of malignant gliomas. Semin Oncol. 1994;21:249-259.

78. Kruse C A, Cepeda L, Owens B, Johnson S D, Stears J, Lillehei K O. Treatment of recurrent glioma with intracavitary alloreactive cytotoxic T lymphocytes and interleukin-2. Cancer Immunol Immunother. 1997;45:77-87.

79. Merchant R E, Baldwin N G, Rice C D, Bear H D. Adoptive immunotherapy of malignant glioma using tumor-sensitized T lymphocytes. Neurol Res. 1997;19:145-152.

80. Nakagawa K, Kamezaki T, Shibata Y, Tsunoda T, Meguro K, Nose T. Effect of lymphokine-activated killer cells with or without radiation therapy against malignant brain tumors. Neurol Med Chir (Tokyo). 1995;35:22-27.

81. Plautz G E, Barnett G H, Miller D W, et al. Systemic T cell adoptive immunotherapy of malignant gliomas. J Neurosurg. 1998;89:42-51.

82. Sankhla S K, Nadkarni J S, Bhagwati S N. Adoptive immunotherapy using lymphokine-activated killer (LAK) cells and interleukin-2 for recurrent malignant primary brain tumors. J Neurooncol. 1996;27:133-140.

83. Sawamura Y, de Tribolet N. Immunotherapy of brain tumors. J Neurosurg Sci. 1990;34:265-278.

84. Thomas C, Schober R, Lenard H G, Lumenta C B, Jacques D B, Wechsler W. Immunotherapy with stimulated autologous lymphocytes in a case of a juvenile anaplastic glioma. Neuropediatrics. 1992;23:123-125.

85. Tsurushima H, Liu S Q, Tuboi K, et al. Reduction of end-stage malignant glioma by injection with autologous cytotoxic T lymphocytes. Jpn J Cancer Res. 1999;90:536-545.

86. Barba D, Saris S C, Holder C, Rosenberg S A, Oldfield E H. Intratumoral LAK cell and interleukin-2 therapy of human gliomas. J Neurosurg. 1989;70:175-182.

87. Hayes R L, Koslow M, Hiesiger E M, et al. Improved long term survival after intracavitary interleukin-2 and lymphokine-activated killer cells for adults with recurrent malignant glioma. Cancer. 1995;76:840-852.

88. Ingram M, Jacques S, Freshwater D B, Techy G B, Shelden C H, Helsper J T. Salvage immunotherapy of malignant glioma. Arch Surg. 1987;122:1483-1486.

89. Jacobs S K, Wilson D J, Komblith P L, Grimm E A. Interleukin-2 or autologous lymphokine-activated killer cell treatment of malignant glioma: phase I trial. Cancer Res. 1986;46:2101-2104.

90. Jeffes E W, III, Beamer Y B, Jacques S, et al. Therapy of recurrent high-grade gliomas with surgery, autologous mitogen-activated IL-2-stimulated (MAK) killer lymphocytes, and rIL-2: II. Correlation of survival with MAK cell tumor necrosis factor production in vitro. Lymphokine Cytokine Res. 1991;10:89-94.

91. Merchant R E, McVicar D W, Merchant L H, Young H F. Treatment of recurrent malignant glioma by repeated intracerebral injections of human recombinant interleukin-2 alone or in combination with systemic interferon-alpha. Results of a phase I clinical trial. J Neurooncol. 1992;12:75-83.

92. Yoshida S, Takai N, Saito T, Tanaka R. [Adoptive immunotherapy in patients with malignant glioma]. Gan To Kagaku Ryoho. 1987;14:1930-1932.

93. Davico B L, De Monte L B, Spagnoli G C, et al. Bispecific monoclonal antibody anti-CD3×anti-tenascin: an immunotherapeutic agent for human glioma. Int J Cancer. 1995;61:509-515.

94. Jung G, Brandl M, Eisner W, et al. Local immunotherapy of glioma patients with a combination of 2 bispecific antibody fragments and resting autologous lymphocytes: evidence for in situ t-cell activation and therapeutic efficacy. Int J Cancer. 2001;91:225-230.

95. Pfosser A, Brandl M, Salih H, Grosse-Hovest L, Jung G. Role of target antigen in bispecific-antibody-mediated killing of human glioblastoma cells: a pre-clinical study. Int J Cancer. 1999;80:612-616.

96. Yoshida J, Takaoka T, Mizuno M, Momota H, Okada H. Cytolysis of malignant glioma cells by lymphokine-activated killer cells combined with anti-CD3/antiglioma bifunctional antibody and tumor necrosis factor-alpha. J Surg Oncol. 1996;62:177-182.

97. Imaizumi T, Kuramoto T, Matsunaga K, et al. Expression of the tumor-rejection antigen SART1 in brain tumors. Int J Cancer. 1999;83:760-764.

98. Eshhar Z, Waks T, Gross G, Schindler D G. Specific activation and targeting of cytotoxic lymphocytes through chimeric single chains consisting of antibody-binding domains and the gamma or zeta subunits of the immunoglobulin and T-cell receptors. Proc Natl Acad Sci USA. 1993;90:720-724.

99. Haynes N M, Snook M B, Trapani J A, et al. Redirecting mouse CTL against colon carcinoma: superior signaling efficacy of single-chain variable domain chimeras containing TCR-zeta vs Fc epsilon RI-gamma. J Immunol. 2001;166:182-187.

100. Hombach A, Heuser C, Sircar R, et al. An anti-CD30 chimeric receptor that mediates CD3-zeta-independent T-cell activation against Hodgkin's lymphoma cells in the presence of soluble CD30. Cancer Res. 1998;58:1116-1119.

101. Hombach A, Schneider C, Sent D, et al. An entirely humanized CD3 zeta-chain signaling receptor that directs peripheral blood t cells to specific lysis of carcinoembryonic antigen-positive tumor cells. Int J Cancer. 2000;88:115-120.

102. Hombach A, Sircar R, Heuser C, et al. Chimeric anti-TAG72 receptors with immunoglobulin constant Fc domains and gamma or zeta signalling chains. Int J Mol Med. 1998;2:99-103.

103. Moritz D, Wels W, Mattem J, Groner B. Cytotoxic T lymphocytes with a grafted recognition specificity for ERBB2-expressing tumor cells. Proc Natl Acad Sci USA. 1994;91:4318-4322.

104. Weijtens M E, Willemsen R A, Valerio D, Stam K, Bolhuis R L. Single chain Ig/gamma gene-redirected human T lymphocytes produce cytokines, specifically lyse tumor cells, and recycle lytic capacity. J Immunol. 1996;157:836-843.

105. Altenschmidt U, Klundt E, Groner B. Adoptive transfer of in vitro-targeted, activated T lymphocytes results in total tumor regression. J Immunol. 1997;159:5509-5515.

106. Jensen M, Tan G, Forman S, Wu A M, Raubitschek A. CD20 is a molecular target for scFvFc:zeta receptor redirected T cells: implications for cellular immunotherapy of CD20+malignancy. Biol Blood Marrow Transplant. 1998;4:75-83.

107. Jensen M C, Clarke P, Tan G, et al. Human T lymphocyte genetic modification with naked DNA. Mol Ther. 2000;1:49-55.

108. Minty A, Chalon P, Derocq J M, et al. Interleukin-13 is a new human lymphokine regulating inflammatory and immune responses. Nature. 1993;362:248-250.

109. Boon T, Cerottini J C, Van den E B, van der B P, Van Pel A. Tumor antigens recognized by T lymphocytes. Annu Rev Immunol. 1994;12:337-365.

110. Castelli C, Rivoltini L, Andreola G, Carrabba M, Renkvist N, Parmiani G. T-cell recognition of melanoma-associated antigens. J Cell Physiol. 2000;182:323-331.

111. Chi D D, Merchant R E, Rand R, et al. Molecular detection of tumor-associated antigens shared by human cutaneous melanomas and gliomas. Am J Pathol. 1997;150:2143-2152.

112. Boon T, Coulie P, Marchand M, Weynants P, Wolfel T, Brichard V. Genes coding for tumor rejection antigens: perspectives for specific immunotherapy. Important Adv Oncol. 1994;53-69.

113. Cebon J, MacGregor D, Scott A, DeBoer R. Immunotherapy of melanoma: targeting defined antigens. Australas J Dermatol. 1997;38 Suppl 1:S66-S72.

114. Greenberg P D, Riddell S R. Tumor-specific T-cell immunity: ready for prime time? J Natl Cancer Inst. 1992;84:1059-1061.

115. Cohen J L, Saron M F, Boyer O. et al. Preservation of graft-versus-infection effects after suicide gene therapy for prevention of graft-versus-host disease. Hum Gene Ther. 2000;11:2473-2481.

116. Drobyski W R, Morse H C, III, Burns W H, Casper J T, Sandford G. Protection from lethal murine graft-versus-host disease without compromise of alloengraftment using transgenic donor T cells expressing a thymidine kinase suicide gene. Blood. 2001;97:2506-2513.

117. Link C J, Jr., Traynor A, Seregina T, Burt R K. Adoptive immunotherapy for leukemia: donor lymphocytes transduced with the herpes simplex thymidine kinase gene. Cancer Treat Res. 1999;101:369-375.

118. Spencer D M. Developments in suicide genes for preclinical and clinical applications. Curr Opin Mol Ther. 2000;2:433-440.

I claim:

1. A chimeric immunoreceptor comprising the following linked elements, arranged in the stated order:
a) an extracellular domain comprising a soluble receptor ligand,
b) a support region capable of tethering the extracellular domain to a cell surface,
c) a transmembrane region, and
d) an intracellular signalling domain.

2. The chimeric immunoreceptor of claim 1, wherein the extracellular domain comprises a soluble ligand selected from the group consisting of autocrine growth factors, paracrine growth factors, chemokines, cytokines, hormones, and engineered artificial small molecule ligands.

3. The chimeric immunoreceptor of claim 1, wherein the support region is selected from the group consisting of the constant regions of immunoglobins, CD8, and artificial linkers.

4. The chimeric immunoreceptor of claim 1, wherein the transmembrane region is a transmembrane domain of a leukocyte CD marker.

5. The chimeric immunoreceptor of claim 1, wherein the intracellular signalling domain is selected from the group consisting of the intracellular receptor signalling domain of the T cell antigen complex, FcγRIII costimulatory domains, CD28, DAP10 and CD2.

6. The chimeric immunoreceptor of claim 1, comprising the following linked elements, in the stated order:
a) IL13(E13Y),
b) an IgG4 constant region,
c) a CD4 transmembrane domain, and
d) an intracellular T cell antigen receptor CD3 complex zeta chain.

7. The chimeric immunoreceptor of claim 1, wherein the immunoreceptor is expressed by a T lymphocyte cell line transformed with a DNA sequence encoding the immunoreceptor.

8. A method for treating human cancer, comprising administering to a human suffering from cancer a plurality of cells expressing an immunoreceptor of any of claims 1-7, wherein the soluble receptor ligand of the immunoreceptor is specific for a cancer-specific cell surface receptor.

9. The method of claim 8, wherein the the cancer-specific cell surface receptor is a cytokine receptor.

10. The method of claim 9, wherein the soluble receptor ligand is IL-13(E13Y).
